(19) United States
(12) Patent Application Publication
(10) Pub. No.: US 2011/0313145 A1
(43) Pub. Date: Dec. 22, 2011

(54) ISOLATION OF RNA

(75) Inventors: Andre Sharon, Newton, MA (US); Anirban Chatterjee, Malden, MA (US); Paul Mirsky, Jamaica Plain, MA (US); Alexis Sauer-Budge, Lincoln, MA (US)

(73) Assignees: FRAUNHOFER USA, INC., Plymouth, MI (US); TRUSTEES OF BOSTON UNIVERSITY, Boston, MA (US)

(21) Appl. No.: 13/157,929

(22) Filed: Jun. 10, 2011

Related U.S. Application Data
(63) Continuation of application No. PCT/US2009/067656, filed on Dec. 11, 2009.
(60) Provisional application No. 61/121,665, filed on Dec. 11, 2008.
(60) Provisional application No. 61/121,688, filed on Dec. 11, 2008.

(51) Int. Cl. C07H 1/08; B01D 35/00; C12M 1/24

(52) U.S. Cl. 536/25.41; 435/306.1; 422/535

(57) ABSTRACT

Disclosed herein are methods for purification of RNA from a sample. The RNA can be total RNA or mRNA. The method involves preparing the sample in a solution of lysis buffer and depositing into a first end of a lysis straw such that the sample solution flows through the matrix of the lysis straw and is eluted from the opposite end of the lysis straw, and depositing the eluted material into a first end of a solid phase extraction (SPE) straw, such that the deposited solution flows through the matrix of the SPE straw towards the opposite end of the SPE straw, and eluting the RNA from the SPE straw by depositing a solution of elution buffer, into the first end of the SPE straw, such that the deposited solution flows through the matrix of the SPE straw and is eluted from the opposite end of the SPE straw, wherein purified RNA from the sample is present in the eluate of the SPE straw. When the RNA is total RNA, and the sample is a cell sample, and step b) requires adding a precipitating solution to the eluted material from step a), and depositing the solution into the first end of the SPE straw, wherein the straw comprises silica microspheres, such that the deposited solution flows through the matrix of the SPE straw toward the opposite end of the SPE straw. When the RNA is mRNA and step b) further comprises depositing the solution into the first end of the solid phase extraction (SPE) straw, wherein the straw comprises oligo-dT. Pressure (e.g., at least 10 psi pressure) and heat (e.g., about 60° C.) can be applied to the samples, and/or eluates and/or straws. Examples of lysis buffers, loading buffers and elution buffers are provided. Also disclosed are the specific straws and porous polymer monolith matrix components.

### RELATED APPLICATIONS

This application claims the benefit under 35 U.S.C. §120 and is a Continuation of International PCT Application No. PCT/US2009/067656 filed Dec. 11, 2009, which claims the benefit under 35 U.S.C. §119(e) of U.S. Provisional Patent Application Ser. No. 61/121,665 filed Dec. 11, 2008 and U.S. Provisional Patent Application Ser. No. 61/121,688 filed Dec. 11, 2008, the contents of which are herein incorporated by reference in their entirety.

### FIELD OF THE INVENTION

The present invention relates to the field of nucleic acid and protein isolation.

### BACKGROUND OF THE INVENTION

RNA is an important bio-molecule involved in the transfer of information from the genetic material of DNA to the functional complexes of proteins in the living cells. The intermediate biomolecules responsible for translating the information content of the DNA to the “executing” protein biomolecules are known as the messenger RNA or mRNA. Almost all changes affecting cells reflected in gene expressions and can be measured at the level of changes in messenger RNA (mRNA). Hence, RNA isolation from eukaryotic cells is a necessary and essential step in sample preparation for gene expression. High throughput gene expression analysis is necessary in the areas of biologic drug discovery, clinical trials in drug development, pharmacogenomics and molecular diagnostics.

RNA isolated from cells can be studied at two levels, either at the total RNA level or at the more specific mRNA level. While mRNA levels are more specific for gene expression analysis, total RNA conditions also reflect large scale changes in the properties of the cell (i.e. cancer or necrosis etc).

The current methods available for isolating total RNA or mRNA from the cells depend upon either a solid phase extraction using surface properties of RNA (Qiagen RNAeasy or Ambion RNAqeuos kit), a polyA tail capture method using oligo-dT attached to cellulose beads (Ambion PolyA Purist and Invitrogen Dynabeads kits) or through magnetic particles (Ambion MagMax kit and Qiagen MagAttract kit). Some of these kits have been modified to be used on semiautomated/automated platforms for medium throughput RNA isolation like Qiagen MagAttract Direct mRNA M48 Kit for the BioRobot M48 Workstation, the semmi automated MagMax 96 kit from Ambion or the fully automated low throughput total RNA isolation iPrep instrument from invitrogen.

RNA isolation from eukaryotic cells is especially challenging due to the fact that RNA comprises only about 2-3% of the total cellular nucleic acid material and is dominated by the amount of genomic DNA present in cells (>96%). Hence any attempt to isolate RNA must be effective against genomic DNA contamination. Further, RNA is degraded by a ubiquitous and very stable class of enzymes called RNAse. RNAse is found in the target tissue/cells as well as in bacteria, fungi, human skin, sputum and other possible contaminating sources. Some of these RNAses are not removed/neutralized by regular sterilization processes. Thus, RNA degradation due to the presence of RNAse is the biggest threat in the RNA isolation process. Use of proper conditions and safeguards and fast handling of the samples are necessary to reduce RNA degradation due to RNAse contamination. Another complication is that mRNA are transitory molecules and the mRNA content of a live cell can easily change during handling. Thus proper handling of a cell is important to minimize artifacts in mRNA expression. Finally, since mRNA is only about 2-4% of the total RNA content in a cell or only about 0.04-0.02% of the total nucleic acid content of a cell, it is imperative to create a sensitive and high yielding RNA isolation process.

Currently several “kits” are available in the market for RNA isolation from a diverse variety of samples. The largest competitors are Qiagen, Ambion (now Invitrogen), Roche and Promega. The kits are variously placed to isolate total RNA or mRNA from cultured mammalian and non mammalian eukaryotic cells, animal tissues, blood and other biological fluids, fungi, plant cells, bacteria etc. The technologies in use are—solid phase extraction, magnetic bead based separation and oligo-dT based isolation. Most of these kits are based on a solid phase extraction technique or the magnetic for isolating nucleic acids. Such processes are non specific for DNA or RNA and the RNA is enriched by either the enzymatic removal of DNA (by DNAse treatment) or by a second set of bead based separation technique for isolating nucleic acids (e.g., by a second set of columns specific for isolatin of mRNA). Also available are the non-kit based RNA isolation reagents like the “Trizol” reagent from Invitrogen, or “Quiazol” from Quiagen, which have higher yield than the “kits”. However these products are hazardous and difficult to automate for high throughput purposes.

The advantage of the nucleic acid isolation kits is their relative ease for automation. Several of such kits or kit based techniques have been adopted for medium throughput automation (up to a few hundred samples a day). However, a significant part of the process is still outside the bounds of the automated instrument (like centrifugation and tissue lysis) and has to be handled offline, thus limiting the scope of the “automation”, and preventing the kits from being used as real high throughput instruments. Also the yield and sensitivity of these instruments are not as good as the manual processes. Currently available kits are listed in Table 1 along with pros and cons. The “automated” versions of such processes are listed in Table 2.

A number of researchers around the world are using Porous Polymer Monoliths (PPM) for chemical separations such as Solid-Phase Extraction (SPE), as well as for cell lysis. For processes involving PPMs, the user typically dispenses fluid through a tube or column filled with the PPM material. Most of these research groups use syringe pumps, which offer very accurate control over flow rate and volume, but are very expensive. Because of their cost and size, it is impractical to gang large arrays of them together to form a high-throughput tool. However, many of the applications of PPMs are best-suited to a high-throughput format. An additional problem is that most existing PPM devices are built in the format of a microfluidic chip, consisting of a patterned chip bonded to a cover layer. While such chips may eventually become very cheap to mass produce, it is fairly time-consuming to produce them on a prototype scale in the laboratory. Meanwhile, researchers studying PPMs need to conduct great numbers of experiments in order to probe the properties of these interesting materials.

There are some precedents for the design used in this machine. One is the vacuum manifold used to suck samples through solid-phase extraction columns. Another is fabrication of PPM columns inside of a pipette tip, which also eliminates the need to fabricate microfluidic chips, and allows one to use the pipettor itself as a simple means of dispensing liquids through the PPM.

### SUMMARY OF THE INVENTION

Aspects of the present invention relate to a method for purification of RNA from a sample, comprising preparing the sample in a solution of lysis buffer and depositing the sample solution into a first end of a lysis straw such that the sample solution flows through the matrix of the lysis straw and is eluted from the opposite end of the lysis straw, and depositing the eluted material into a first end of a solid phase extraction (SPE) straw, such that the deposited solution flows through the matrix of the SPE straw towards the opposite end of the SPE straw, and eluting the RNA from the SPE straw by depositing a solution of elution buffer, into the first end of the SPE straw, such that the deposited solution flows through the matrix of the SPE straw and is eluted from the opposite end of the SPE straw, wherein purified RNA from the sample is present in the eluate of the SPE straw. In one embodiment, the RNA is total RNA, the sample is a cell sample, and step b) requires adding a precipitating solution to the eluted material from step a), and then depositing the solution into the first end of the solid phase extraction (SPE) straw, wherein the straw comprises silica microspheres, such that the deposited solution flows through the matrix of the SPE straw towards the opposite end of the SPE straw. In one embodiment, the RNA is mRNA, and step b) further comprises depositing the solution into the first end of the solid phase extraction (SPE) straw, wherein the straw comprises oligo-dT.

In one embodiment of the above described methods, at least 10 psi pressure is applied to the lysis straw and/or to the SPE straw at the first end of the straw, following solution deposition, to facilitate movement of the solution through the straw. In one embodiment of the above described methods, from about 10-60 psi pressure is applied to the lysis straw and/or to the SPE straw at the first end of the straw, following solution deposition, to facilitate movement of the solution through the straw. In one embodiment of the above described methods, from about 10-20 psi pressure is applied to the lysis straw and/or to the SPE straw at the first end of the straw, following solution deposition, to facilitate movement of the solution through the straw. In one embodiment of the above described methods, the sample solution and the lysis straw is heated to about 60° C. and/or the eluted material from step a) is heated to about 60° C. for about 10 minutes, and/or the eluted material from step a) is incubated with DNAse in 1×DNAse buffer for 5-10 minutes at about 25° C., and/or the lysis straw eluate solution in step b) is heated to about 60° C. prior to deposition in the SPE straw.

In one embodiment of the above described methods, the lysis buffer is 0.1% Triton X-100, 10 mM Tris-Cl, (pH 7.5), 1 mM EDTA, 1×RNAse inhibitor. In one embodiment of the above described methods, the loading buffer is 500 mM NaCl, 10 mM Tris-Cl (pH 7.5), 1 mM EDTA, 1×RNAse inhibitor. In one embodiment of the above described methods, the elution buffer is 10 mM Tris-Cl, (pH 7.5), 1 mM EDTA, 1×RNAse inhibitor. In one embodiment of the above described methods, the precipitating solution of step b) is an alcohol/salt solution. In one embodiment, the alcohol salt solution of step b) is isopropanol and ammonium acetate pH 5.2, and wherein a volume of isopropanol that is equal to the volume of the eluted material of step a) is added, and a volume of 3M ammonium acetate pH 5.2 that is ⅕^{th} the volume of the eluted material of step a) is added.

In one embodiment of the above described methods, the precipitating solution of step b) is selected from the group consisting of isopropanol, ethanol, sulfolene, butanol, acetone, acetonitrile, and high salt.

In one embodiment of the above described methods, following step b), and prior to step c) 70% Ethanol is added to the SPE straw and air is then passed through the straw at 60 psi.

Aspects of the present invention further relate to an apparatus for purification of total RNA comprising, a lysis porous polymer monolith matrix contained within a first open tube, and a solid phase extraction (SPE) porous polymer monolith matrix comprising silica microspheres contained within a second open tube, the open tubes and the matrixes being able to withstand air pressure of up to 150 psi and temperatures of up to 70° C. In one embodiment, the lysis porous polymer monolith matrix has a pore size of about 3-10 micrometers, and has —COOH functionalized carbon nanotubes embedded within, and/or the SPE porous polymer monolith matrix has a pore size of about 1-5 microns, and has clusters of silica microbeads of 0.70 um embedded within.

Aspects of the present invention further relate to an apparatus for purification of mRNA comprising, a lysis porous polymer monolith matrix contained within a first open tube, and solid phase extraction (SPE) porous polymer monolith matrix comprising oligo dT, contained within a second open tube, the open tubes and the matrixes being able to withstand up to 150 psi air pressure and temperature up to 70° C. In one embodiment, the lysis porous polymer monolith matrix has a pore size of about 3-10 micrometers, and has —COOH functionalized carbon nanotubes embedded within, and/or the SPE porous polymer monolith matrix has a pore size of about 1-5 microns, and contains oligo-dT cellulose.

In one embodiment of the above described inventions, the open tubes are made of a polyolefin and/or have an outer diameter of about 3.18 mm and an inner diameter of 2 mm on mm, and/or a length of about 10 cm. In one embodiment of the above described inventions, the first open tube and the second open tube are each connected at one end to a reservoir for delivery of solution or air to the tube. In one embodiment of the above described inventions, the first open tube and the second open tube are part of the experimental research enabling multichannel air-pressure driven fluid dispenser (ERMAF).

Aspects of the invention further relate to a lysis straw comprising a lysis porous polymer monolith matrix contained within an open tube, wherein the porous polymer monolith matrix has a pore size of about 3-10 micrometers. In one embodiment, the lysis porous polymer monolith matrix has —COOH functionalized multiwall hollow carbon nanotubes, with an outer diameter of 15±5 nm, and an length from 5-20 microns, embedded within.

Aspects of the invention further relate to a solid phase extraction (SPE) straw comprising a solid phase extraction porous polymer monolith matrix contained within an open tube, wherein the porous polymer monolith matrix has a pore size of about 1-5 microns, and has clusters of silica microspheres of 0.70 μm embedded within.

Aspects of the invention further relate to a solid phase extraction (SPE) straw comprising an oligo-dT porous polymer monolith matrix contained within an open tube.

In one embodiment of the above described inventions, the open tube and the porous polymer monolith matrix of the lysis straw or SPE straw is capable of withstanding applied air pressure of up to 100 psi and temperatures of up to 70° C. In one embodiment the open tube is made of a polyolefin and has an outer diameter of about 3.18 mm and an inner diameter of 2 mm or 1 mm and has a length of about 10 cm.

### BRIEF DESCRIPTION OF THE DRAWINGS

FIG. 1 is a photograph of total RNA prepared from MDCK cells by the methods disclosed herein, fractionated by size. Lanes A and C—MDCK RNA from Straws. Lane B—Negative Control. Lanes D and E—MDCK RNA from Ambion RNAqueous Micro Kit. Lanes F and G—MDCK RNA from Ambion RNAqueous Micro Kit with DNAse treatment.

FIG. 2 is a photograph of total RNA prepared from yeast, by the methods disclosed herein, fractionated by size. Lanes A and C—Yeast RNA from Straws. Lane B—Negative Control.

FIG. 3 is a set of two photographs, of an SPE straw (A), and the SPE porous polymer monolith (B) contained therein.

FIG. 4 is a set of three photographs of the straw driver instrument.

FIG. 5 is a schematic of a portion of the straw driver instrument.

FIG. 6 is a schematic of a portion of the straw driver instrument.

FIG. 7 is a schematic of the straw driver instrument, viewed from above.

FIG. 8 is a schematic of the straw driver instrument, viewed from below.

FIG. 9 is a schematic of the straw driver instrument, viewed from the side.

FIG. 10 is set of two photographs, of the lysis straw (A), and the lysis porous polymer monolith contained therein (B).

FIG. 11 is a photograph of the results of a PCR assay performed on cDNA synthesized from purified total RNA prepared from the methods disclosed herein, fractionated by size. Lane 7: 100 bp DNA ladder. Lanes 1 and 2: PCR from cDNA—sample 1. Lanes 2 and 3 sample 2. Lanes 5 and 6: sample 3. Lane 8: negative controls—sample 1. Lane 9: sample 2. Lane 10: sample 3. The arrow indicates the band of interest, present in samples, absent in negative controls.

FIG. 12 is a photograph of the results of a PCR assay performed on cDNA synthesized from purified total RNA prepared from the methods disclosed herein, fractionated by size. Lane 1: 100 bp DNA ladder. Lane 2: PCR from cDNA—sample 1. Lane 3: negative controls—sample 1. Lane 4: sample 2. Lane 6: sample 3. Lane 7: sample 3. Lane 8: PCR negative control. Lane 9: PCR positive control. The arrow indicates the band of interest, present in samples, absent in negative controls.

FIG. 13 is a photograph of a PCR assay performed on cDNA synthesized from purified mRNA prepared from the methods disclosed herein, fractionated by size. Lanes 1 and 8: 100 bp DNA ladder. Lanes 2 and 3 PCR from cDNA—sample 1. Lanes 4 and 5: sample 2. Lanes 6 and 7: sample 3. Lane 9: negative controls—sample 1. Lane 10: sample 2. Lane 11: sample 3. Arrow indicates the band of interest, present in samples, absent in negative controls.

FIG. 14 is a bar graph of data obtained from the quantitation of total RNA obtained from a Qiagen RNA isolation kit, versus samples obtained from the Qiagen RNA isolation kit, repurified by the methods described herein.

FIG. 15 is a bar graph of data showing the average time to flow 200 μl of various reagents through the PPM/SPE straws described herein.

FIG. 16 is a graphical representation of the amounts of total RNA obtained from the indicated amounts of MDCK cells by the Ambion kit method, versus the methods described herein for the purification of total RNA.

FIG. 17A-FIG. 17G is a schematic of the generation of straws for RNA purification.

### DETAILED DESCRIPTION OF THE INVENTION

The isolation of RNA from mammalian cells is an important and essential step for many of the life science research activities, especially with relation to biologic drug discovery, clinical trials and pharmacogenomics. The existing protocols and methods are typically at best suited for low to medium throughput analyses and there can be improvement in terms of yield, speed and throughput levels. In the present invention, we present a new method by which RNA (e.g., mRNA and total RNA) can be isolated from a sample (e.g., mammalian cells) faster, cheaper and at a higher yield than generally available techniques. The new method is faster since it requires very little human intervention, no offline steps like centrifugation or DNase treatment. The costs involved are low since the method requires almost no hazardous chemicals and cheap “disposable” components. The high yield also makes ensures the requirement of very little sample, adding to cost savings in cell culture or sample storage. Even more importantly, the methods and apparatus presented are ideally suited for high throughput automation to analyze large number of samples.

One aspect of the present invention relates to an apparatus designed for the purification of RNA (total or mRNA) from an organism or sample (mammalian cells, organ, tissue, bodily fluid or extract, yeast cells, fungi, bacterial cells, etc.). The apparatus comprises two tubes, with openings at either end (referred to herein as straws), that contain within them porous polymer monolith matrixes. The matrixes contained therein contact the walls of the straw to the extent that any sample deposited within the straw at one end, must pass substantially through the matrix in order to exit the straw at the other end. One of the straws, referred to herein as the first straw, is a lysis straw, and contains a lysis porous polymer monolith matrix. The other straw, referred to herein as the second straw, is a solid phase extraction (SPE) straw and contains an SPE porous polymer monolith matrix.

For the purification of total RNA, the SPE porous polymer monolith matrix comprises silica (e.g., silica microspheres) or some other polymer that generally binds nucleic acid. For the purification of mRNA, the SPE porous polymer monolith matrix comprises a component that selectively binds the poly(A) RNA (e.g., an oligo-dT porous polymer monolith matrix).

Another aspect of the present invention relates to a method for purification of total RNA from an organism or sample (mammalian cells, organ, tissue, bodily fluid or extract, yeast cells, fungi, bacterial cells, etc.). The method utilizes one or more components of the apparatus described herein. Solid phase extraction allows for both the purification and preconcentration of biological samples. The methods of the present invention utilize immobilized particles which bind the desired biological molecule (e.g. total RNA, mRNA, protein, etc.) in a porous polymer monolith to form a microscale solid-phase extraction system. In the method, the sample (e.g., cell sample) is prepared in a solution of lysis buffer. It is then deposited into the receiving end of the lysis straw. The deposited sample is then made to flow through the matrix of the lysis straw (e.g., through the application of pressure) and is eluted from the opposite end of the lysis straw (the eluting end). Heat is optionally applied to the solution and/or the lysis straw.

For the purification of total RNA, a precipitating solution (e.g, an alcohol/salt solution) is then added to the eluted material. The eluted material with the precipitating solution is then deposited onto the receiving end of a solid phase extraction straw. The deposited solution is then made to flow through the matrix of the SPE straw and is eluted from the opposite end. The eluate contains highly purified, intact total RNA from the cell sample, in a relatively high concentration.

For the purification of mRNA, the eluted material is prepared in a solution of loading buffer suitable for selective binding of poly(A) RNA with poly(U) or poly(T) nucleic acid molecule, and then deposited onto the receiving end of a solid phase extraction straw. The deposited solution is then made to flow through the matrix of the SPE straw and is eluted from the opposite end. The eluate contains highly purified, intact mRNA from the cell sample, in a relatively high concentration.

Another aspect of the present invention relates to the straw containing the porous polymer monolith matrix, described herein (e.g., lysis straw, SPE straw). Another aspect of the present invention relates to an apparatus that is designed to contain one or more of the straws containing the porous polymer monolith matrix, described herein. The apparatus may optionally be set up for the application of heat and/or pressure to the straws and/or samples applied or eluted from the straws.

The isolation procedures (e.g., RNA) and materials required for their performance, disclosed herein can easily be fabricated at the laboratory scale for research purposes. The use of the straw system and a variation of the protocol can be used to isolate genomic DNA from bacterial, yeast and mammalian cells. Another variation can be used to isolate plasmid DNA from bacterial cells. The use of the invention described herein can facilitate screening for candidate drug molecules for clinical trials, discovery of potential drug targets in the cells by looking at up or down regulation of genes during treatment or cell stress, molecular diagnostics through DNA based microarray, and high throughput pharmacogenomics. Throughput for the apparatus and methods described herein is more than 400 samples, and can be more than 500 samples, and can even be greater than 600 samples from which the biological material (e.g., RNA) is isolated/purified. Typical yields of RNA (total or mRNA) obtained are 80-90%, with potential yields being 99% of the total or mRNA, respectively, in the original sample.

### Lysis Buffers

In one embodiment, a gentle lysis buffer is used to isolate total RNA. One such gentle lysis buffer is 0.1% Triton X-100, 10 mM Tris-Cl, (pH 7.5), 1 mM EDTA, 1×RNAse inhibitor. Other non-ionic detergents like Tween-20, Tween-80, Triton X-114, NP40 etc can be used instead of TritonX-100 for “gentle lysis” of the cells. Chaotrophic buffers like Guanidium thiocyanate or Urea instead of TritonX-100 in the lysis buffer can be used to provide stronger lysing (e.g., for tougher cells such as yeast). Chaotrophic buffers can also be used for total lysis of cells to isolate nuclear RNA from mammalian cells. Strong ionic detergents like sodium lauryl sulphate or sodium lauryl sarcosyl maybe used instead of chaotrophic buffers for total lysis of cells.

### Elution Buffer

Elution buffer is used to elute the bound RNA from the SPE straw monolith matrix. One such elution buffer is 10 mM Tris-Cl, (pH 7.5), 1 mM EDTA, 1×RNAse inhibitor. These components can be substituted with like components to produce alternate elution buffers, (e.g, HEPES buffer can be substituted for TE).

Other variations on the various disclosed buffers (e.g., lysis and elution) are expected to also function in the methods of the instant invention, provided they have similar desired properties (e.g., ionic strength, pH, etc.). The salt and pH in the various buffers can be optimized for the specific ribonuclease inhibitors (RNAse inhibitor) being used as well.

Various ribonuclease inhibitors are available, including, without limitation, RNAsin® (Promega), StopRNase inhibitor (SPRIME Gmbl), ANTI-RNase (Ambion), and can be used in adapted versions of the methods describd herein.

### Precipitating Solutions

In one embodiment, a precipitating solution is further added to the eluate of a lysis straw. Such a solution is, for example, an organic solvent solution. In one embodiment, the precipitating solution is an alcohol/salt solution. Various alcohols (e.g., ethanol, methanol, butanol, propanol, etc.) and salts (e.g., ammonium acetate, sodium chloride, sodium acetate, lithium chloride, guanidine thiocyanate, etc.) are useful in the invention. In one embodiment, the salt is a chaotropic salt. One example of such a solution is isopropanol and ammonium acetate pH 5.2. In one embodiment, a single volume of isopropanol is added to the sample, and ⅕^{th} volume of 3M ammonium acetate pH 5.2 is added. In one embodiment, the amount of alcohol/salt solution added is sufficient to precipitate the nucleic acids. Adjustment to the alcohol and salt used may also require an adjustment of the amount used.

Other organic solutions known in the art may also be used for precipitation of the RNA. Other such solutions include, without limitation, sulfolene, acetone, and acetonitrile. A high salt concentration (e.g., >200 mM) can be used as well. Combinations of the solvent solutions can be used as well.

In one embodiment, the organic solvent (e.g, ethanol, acetone, acetonitrile and isopropanol) is added to a final concentration of from about 40% to about 80%. One range is about 45% to about 75%, and all concentrations in between, for example, 45, . . . 50, . . . 55, . . . 66, . . . 70. In one embodiment, the final concentration is about 66%. In one embodiment, sulfolene is added to a final concentration of about 45%. Useful salt concentrations for precipitating are from about 100 mM to 1000 mM (1M).

### Washes

The straws are optionally washed at any given step. This may not be necessary for a variety of isolation methods. But, if desired, alcohol can be used (e.g., 70% alcohol) or water (e.g., DEPC treated water). Alternatively, any of the buffers described herein can be used (e.g., lysis buffer, elution buffer).

### Pressure and Heat Application

The samples once applied to the straws flow through the matrixes. In one embodiment, air pressure is applied to the straws to facilitate sample flow (e.g., 50 psi). This can occur by applying air pressure to the receiving end of the straw. Pressure applied is preferably high (e.g., at least 10 psi), and can range from 10-50, 10-60, 10-70 psi, 10-80, 10-90, etc. Other ranges can be at least 20 psi, at least 25 psi, at least 30 psi, at least 40 psi, at least 50 psi. Up to 200 psi can be applied to the sample to promote appropriate flow and isolation of the desired biomolecules from the sample.

Heat may also be applied to the samples to facilitate purification of the samples and/or degredation of RNAses. The straws can also be heated during the purification process. In purification of RNA, the samples will include an RNAse inhibitor. The amount of heat will depend upon the requirements of the RNAse inhibitor (e.g., 60° C.±3° C. for RNAsecure). In some embodiments, the heat is about 50° C., 60° C., 70° C., 80° C., 90° C., or 100° C.±3° C. or ±5° C. In one embodiment, the cell sample in the lysis buffer and the lysis straw is heated (e.g., about 60° C.±3° C.). The eluted samples (e.g., from the lysis straw and/or the SPE straw) can further be heated (e.g., about 60° C.±3° C.). In one embodiment, a combination of pressure and heat is applied to the sample in the lysis straw (e.g., the sample and straw are heated to about 60° C.±3°) and pressure of about 50 psi is applied).

### Porous Polymer Monolith

The porour polymer monolith matrix contained within the straws is designed for the specific function of the straw (e.g., gently cell lysis or nucleic acid purification). Pore size will vary depending upon the biomolecular components being purified. Content of the matrix will also vary depending upon the biomolecule to be purified. For instance, silica beads can be incorporated for general binding of nucleic acids, oligo-dT can be incorporated for specific binding of poly-A mRNA. Generation of porous polymer monolith matrixes known in the art (e.g., Klapperich et al., US 2007/0015179; Klapperich et al., U.S. Provisional Application 60/921,404, filed Apr. 2, 2007; Klapperich et al., PCT/US08/59158; Xie et al., Advances in Biochemical Engineering/Biotechnology 76: 88 (2002); Hentze et al., Reviews in Molecular Biotechnology 90: 27-53 (2002); West et al., Methods in Molecular Biology 385: 9-21 (2007); West et al., US 2005/0095602; Frechet et al., US 2004/0101442; the contents of each of which are incorporated by referenced herein in their entirety) and can be adapted for use in the inventions described herein by the skilled practitioner.

In one embodiment, the porous polymer monolith is grafted onto the container (e.g., the straws). In this process, the substrate of the container is surface modified prior to the formation of the monolith to improve adhesion of the monolith. This can be accomplished through the use of a grafting polymer. With many substrates, such as thermoplastic polymers, the surface can be modified by the use of photografting with a thin interlayer polymer prior to the preparation of the monolith in the container. This can be achieved by, for example, photografting the inner surface with ethylene diacrylate (EDA) through UV-initiated reactions mediated by benzophenone. For example, one can coat the tube with a mixture of EDA and a hydrogen abstracting photoinitiator, such as 3% benzophenone. The tube can then be UV-irradiated for suitable time, for example, about 1-5 minutes, preferably 3 minutes. The grafting step can be carried out such that it leads to very low conversion. In one embodiment, this avoids the formation of crosslinked polymer. The excess monomer can optionally be removed from the tube/straw by rinsing. Rinsing can be performed, for example, with methanol.

The monolith is formed by polymerization of a mixture of monomers (e.g., EDMA and BuMA). Porogenic solvents can be added to the polymerization mixture to make the polymer monolith permeable. The porogenic solvents dissolve all the monomers and initiator to a form a homogeneous solution. The amount and type of porogen are controlled, and the phase separation process during polymerization leads to the desired pore structure (Yu, C., et al., Anal Chem 73, 5088-96 (2001)). Convenient pore sizes for a lysis porous polymer monolith matrix is about 3-10 micrometers. Convenient pore sizes for SPE porous polymer monolith matrix is about 1-5 microns (e.g., and can have clusters of silica microbeads or oligo dT cellulose embedded within).

### Lysis Porous Polymer Monolith Matrix

In one embodiment, the porous polymer matrix is made with embedded particles which serve as mechanical obstacles to help to lyse the cells of the sample deposited thereon. Nanotubes, typically carbon nanotubes, about 1-50 microns, preferably about 1-20 microns, or 1-10 microns long and about 10-300 nm in diameter are used. Preferably about 30-150 nm, but alternatively about 50-150 nm in diameter are used. The resulting open pore structure contains exposed nanotubes which lyse cells which contact the monolith. Such nanotubes have been used in the art for bacterial lysis (Srivastava, A., et al., Carbon nanotube filters. Nat Mater, 2004. 3(9): p. 610-4; Valcarcel, M., et al., Present and future applications of carbon nanotubes to analytical science. Anal Bioanal Chem, 2005. 382(8): p. 1783-90). In one embodiment, the nanotubes are functionalized (e.g., with —COOH).

### SPE Porous Polymer Monolith Matrix

In one embodiment, the porous polymer monolith matrix is for solid phase extraction (SPE). One such SPE monolith contains silica microbeads/micro spheres. In another embodiment, the SPE monolith contains another hydrophilic surface (e.g, polymer) known to bind nucleic acids. Examples of such hydrophilic surfaces suitable for use in practicing the invention include nitrocellulose, celite diatoms, silica polymers, glass fibers, magnesium silicates, silicone nitrogen compounds (e.g., SIN₄), aluminum silicates, and silica dioxide. The variety of forms that the hydrophilic surfaces can take are also suitable for use in the invention. Suitable forms of hydrophilic surfaces include beads, polymers, particles, etc. In one embodiment, the microbeads (silica or polymer) diameters are from about 150 nm to about 5 μm. In one embodiment, the microbeads are from about 700 nm to about 3000 nm (e micrometers). In one embodiment, the microspheres are 0.70 μm. Nucleic acids will bind to silica in the presence of a high concentration of chaotropic salt. The extracted nucleic acids can be subsequently eluted in an aqueous low-salt buffer, and if necessary, further concentrated into an even smaller volume. The silica beads can also be further functionalized to promote binding of other biomolecules, if desired. The generation of suitable SPE porous polymer monoliths for use in the invention is described herein and further in Klapperich et al., (US 2007/0015179).

mRNA

There are generally three types of RNA molecules: messenger RNA (mRNA), transfer RNA (tRNA), and ribosomal RNA (rRNA). The study of RNA occupies a central role in molecular biology. This molecule performs many different functions in the cell. mRNA, which conveys information from the nucleus to the cytoplasm, is the most intensely studied. Several molecular biology procedures use purified mRNA as starting material. These procedures include: cDNA synthesis (for library construction, RT-PCR analysis, or 5′ end analysis through primer extension); Northern blot analysis; ribonuclease protection assays; and screening procedures involving in vitro translation. There are several existing procedures to purify RNA from various biological samples. However, mRNA represents only 1-5% of the mass of total RNA (Sambrook, 2001). Of the remainder, the major RNA species is ribosomal RNA (rRNA), constituting 80% or more of total RNA mass (Sambrook et al., 1989 and 2001). Although the total RNA isolated from cells can sometimes be used for the above-mentioned procedures, usually a preliminary purification of mRNA from total RNA is often preferred, if not required. This is especially true if the particular mRNA being sought or targeted is in low abundance (0.5% or less of the mRNA population). The presence of rRNA can interfere in the detection of mRNA by Northern blotting, RNase protection assays, differential display analysis, and expression profiling by gene arrays, especially if the target being analyzed is in low abundance. Often, the mRNA from scientifically interesting genes fall into this category.

mRNA can be isolated based on the content of a polyA tail. The porous polymer monolith matrix can be generated to contain a component that selectively binds the poly(A) RNA. The component may be a nucleic acid molecule, RNA or DNA, comprising contiguous “T” and/or “U” residues (“poly(T) or poly(U) nucleic acid molecule”). The RNA and the component are incubated under conditions that allow poly(A) RNA to hybridize with the poly(T) or poly(U) nucleic acid molecule.

It may be useful to add an isostabilizing agent (e.g., an ion or salt) to facilitate the interaction between the poly(A) of the RNA and the component (e.g., in the loading buffer). An isostabilizing agent such as an ion or salt. An isostabilizing agent refers to a solute that affects the hybridization between complementary regions of nucleic acids in such a way that, at certain concentrations of the solute, they negate the difference in the hydrogen bonding between adenine-thymine/uridine (A-T/U) and guanine-cytidine (G-C) base pairs. In some embodiments of the invention tetramethylammonium (TMAC) or tetraethylammonium chloride (TEAC) is specifically contemplated as the isostabilizing agent. In another embodiment betaine is the isostabilizing agent. In still further embodiments, the isostabilizing agent is triethylamine hydrochloride, quinuclidine hydrochloride, or 1,1′-spirobypyrrolidnium bromide. The isostabilizing agent may be added to a sample directly or it may be provided to the sample as a solution containing other components. The isostabilizing agent may be in the form of a binding solution (which facilitates hybridization between the targeted nucleic acid and the component that selects the targeted nucleic acid), wash solution, or elution solution. Other components of the solutions may include buffers, water, detergents, and other salts as described herein. In some embodiments, it is specifically contemplated that certain anions, particularly chaotropic anions, are absent from any solution.

In some embodiments of the invention, the concentration of the isostabilizing agent in a composition comprising a sample and a poly(T) nucleic acid is between about 1.0 M and about 3.0 M, between about 1.2 M and about 2.4 M, or between about 1.5 M and about 2.0 M. In some embodiments, the concentration of the isostabilizing agent in a composition comprising a sample that is incubated under hybridization conditions is about 2.0 M. As discussed above, the isostabilizing agent may be provided to the sample as a binding solution; the concentration of the isostabilizing agent in the binding solution will allow for the concentration of the isostabilizing agent in the composition comprising the sample to be in the ranges discussed above. In some embodiments, the concentration of the isostabilizing agent in a binding solution is “X” times greater than the concentration needed in the composition comprising the sample. Thus, in an embodiment in which the concentration of the isostabilizing agent in composition comprising the sample is about 2.0 M, a 2×. binding solution comprises an isostabilizing agent in a concentration of about 4.0 M, a 3×. binding solution comprises an isostabilizing agent in a concentration of about 6.0 M, etc. Thus, in some embodiments of the invention, a component is provided to the sample in a concentrated form that allows it to be diluted so as to achieve the proper final concentration so as to achieve a desired effect.

A poly(T) or poly(U) nucleic acid molecule refers to a nucleic acid composed of either RNA or DNA, though a poly d(T) nucleic acid molecule is specifically contemplated. Embodiments discussed with respect to a poly d(T) nucleic acid molecule apply with respect to a poly(T) RNA nucleic acid as well. Such a nucleic acid molecule comprises 1) at least 50% “T” and/or “U” residues across the entire molecule, though a nucleic acid molecule comprising greater than 70% or 100% “T” and/or “U” residues is specifically contemplated as part of the invention or 2) comprises a stretch of contiguous “T” and/or “U” residues of at least 14 nucleobases, though a stretch of at least 25, 50, or 100 contiguous “T” and/or “U” residues is specifically contemplated. The use of a poly(U) nucleic acid molecule may be used in place of a poly(T) nucleic acid and vice versa. As discussed above, other components instead of a poly(T) or poly(U) nucleic acid molecule may be employed to target a subset of poly(A) RNA molecules or non poly(A) RNA molecules; such components will similarly use hybridization to isolate such molecules.

In some embodiments of the invention, a composition comprising a sample, poly(dT) nucleic acid molecule, and an isostabilizing agent may first be heated at a temperature between about 60° C. and about 90° C., or between about at least 70° C. and about 90° C., prior to incubation under hybridization conditions. Temperatures of about 60° C., 70° C., 80° C., and 90° C. are specifically contemplated. In some embodiments, hybridization conditions comprise incubating the composition between about 15° C. and 50° C. for at least 3 minutes to 48 hours, or at least 10 minutes to 48 hours, though longer times are contemplated insofar as substantial RNA degradation does not occur. In additional embodiments, incubation time for hybridization is at least 20 minutes, 1 hour, 4 hours, or 8 hours. During hybridization or binding, the sample may be gently rocked. Furthermore, in some embodiments, the binding solution or a solution containing an isostabilizing agent is discarded and additional solution added to the sample; this may be done multiple times.

A poly(dT) or poly(U) nucleic acid may be a synthetic oligonucleotide. In some embodiments, the oligonucleotide is linked, covalently in some embodiments, or physically attached to a non-reacting structure. In other cases, the oligonucleotide is labeled with a compound that reacts with a second compound physically or covalently linked to a non-reacting structure. A “non-reacting structure” refers to a substance that does not chemically react with the targeted molecule—mRNA—so it can be used to aid in separating the targeted molecule from non-targeted molecules. In some embodiments the non-reacting structure is immobilized. In other embodiments, the non-reacting structure is a bead, cellulose, particulate solid, or other matrix. The bead used may be glass or plastic, and it may be synthetic

### Samples

The sample may be any composition that contains RNA (e.g., total and/or mRNA), including tissue or cell lysate, which refers to a composition of substances from the lysis of cells (or cells from a tissue). Cells can be from a multicellular organisms or a single cellular organism, and can be from a single cell type, or can include multiple cell types, they can be derived from one or more tissues from a multicellular organisms (e.g., a subject such as a human or other animal). Methods of the invention may further comprise steps for preparing a tissue lysate for subsequent RNA isolation using the method described herein. The use of lysate preparation protocols is specifically contemplated, including the use of guanidinium isothiocyanate for preparing a lysate.

The term “subject” refers to an animal, for example a human or other mammal. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs, and other animals useful in medical and basic science research. Also included are non-mammalian organisms, such as insects (e.g., drosophila), fish, amphibians, and avians.

### Straws

The straws/tubes used to hold the porous polymer monolith can be made of any material that meets all the manufacturing requirements of the porous polymer monolith (e.g., good mechanical properties, low autofluorescence and high UV transmission) and also meets the requriements of the purification methods described herein (e.g., withstand high pressure and heat). Depending upon those requirement, silicon and/or glass may be an option. Another less expensive option is a polymeric material, such as a cycylic polyolefin. In one embodiment, ZEONEX® or ZEONOR® (e.g. 690R) is used (Zeon Chemicals Inc. Louisville, Ky., USA) or Zeon Corporation (Japan). Other potential substrates include, without limitation, poly(methyl methacrylate), poly(butyl methacrylate), poly(dimethylsiloxane), poly(ethylene terephthalate), poly(butylene terephthalate), hydrogenated polystyrene, polyolefins such as, cyclic olefin copolymer, polyethylene, polypropylene, and polyimide. Polycarbonates and polystyrenes may not be transparent enough for efficient UV transmission and therefore may not be suitable for use as substrates.

In general, the straws are made of a substance that is compatible with generation of the porous polymer monolith matrixes contained therein. Since the straws serve as substrate for monolith deposition, they are considered to be a substrate for the monolith generation. As such, they are preferably resistant to any degredation by the monolith components. Further, if UV light to be used in monolith generation, it is preferably that the straws have certain optical properties, such as light transparency at the desired wavelength range since the photografting reactions must occur within the straws on all sides and the light must first pass through a layer of the substrate polymer. They may further produce low background fluorescence. In one embodiment, the substrate materials is transparent in a wavelength range of 200 to 350 nm, preferably at any point in the range between 230-330 nm such as 250 to 300 nm, 260 to 295, etc.

In one embodiment, the straws are able to withstand application of air pressure through the opening of up to 200 psi. In one embodiment, the straws are able to withstand air pressure of up to 100 psi, or of up to 50-60 psi. In one embodiment, the straws are able to withstand temperatures of 60° C.±5° C. In one embodiment, the straws can withstand temperatures of up to 70° C.±5° C. In one embodiment, the straws can withstand these temperatures in combination with the applied pressure. In one embodiment, the straws are made up of a polyolefin material (e.g., Zeonor 690R Zeon corporation, Japan).

The straws are generally cylindrical, although not necessarily exactly so, and are open at each of the two ends of the cylindrical shape. The exact dimensions of the straws can vary with the different applications of the invention (e.g., amounts to be purified, desired volumes of eluate, specific matrixes, samples, etc.). In one embodiment, the dimensions of the straws are designed to reduce the volumes necessary for the extraction/purification process. As such, they are relatively small. The outer diameter can range from about 2 mm to 5 mm, although it can be enlarged even further (e.g., 6, 7, 8, 9, or 10 mm) to allow for larger sample sizes and larger preparations of purified material. The outer diameter is from about 2 mm to 6 mm, although it also can be enlarged even further (e.g., 7, 8, 9, 10, or 11 mm) to allow for larger sample sizes and larger preparations of purified material. The length of the straws is from about 5 cm to about 15 cm (e.g, 5, 6, 7, 8, 9, 10, 11, 12, 13, 14 or 15 cm) although that also can be increased even further (e.g., up to 20 cm). In one embodiment, the outer diameter is about 3.18 mm and the inner diameter is either 2 mm or 1 mm, and the length of the straws are about 10 cms.

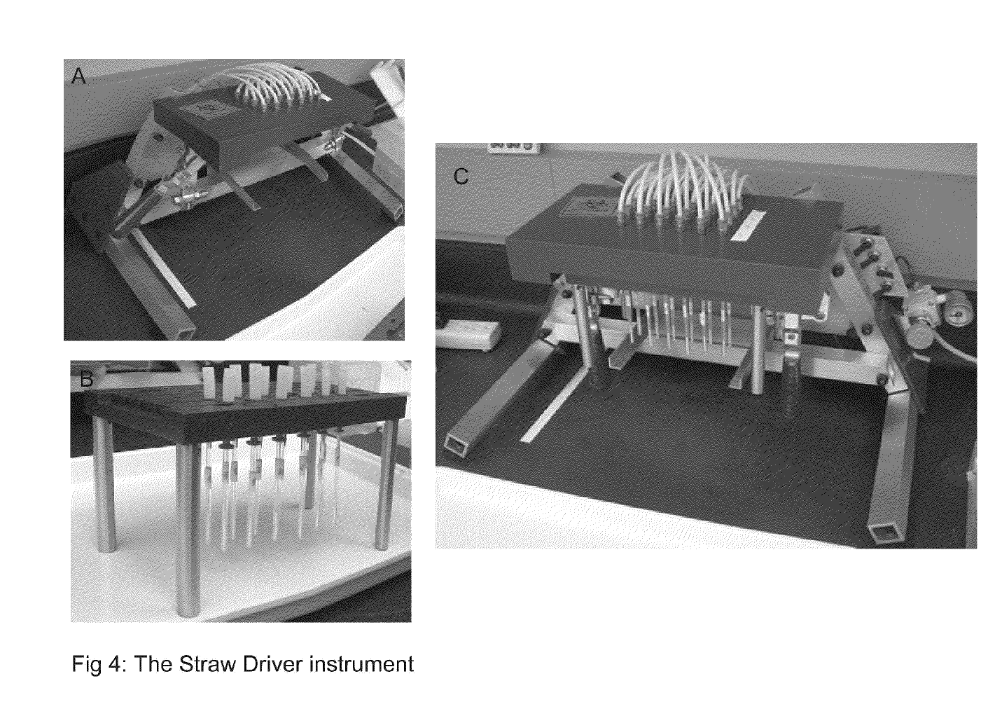

In one embodiment, a straw (e.g., a lysis straw or SPE straw) is situated on a stand to stabilize the straw in the proper orientation for receiving sample. In one embodiment, the straw is attached at one opening (referred to herein as the top or first opening, or receiving end, which serves as end of the straw to receive sample, buffer, etc.) to a reservoir such as a flexible tube through which sample, buffer, or air an be deposited into the straw. In one embodiment, a plurality of identical straws (e.g., either lysis straws or SPE straws) are situated similarly or identically together on a stand for collective, simultaneous use (e.g., FIG. 4B). In one embodiment, the straws are situated on a straw driver instrument, similar to that shown in FIGS. 4 (A and C).

In one embodiment, the reservoir of the SPE straw is attached to the eluting end of the lysis straw (the end from which sample, buffer, etc. is removed or eluted), so that the eluate of the lysis straw can be directly introduced into the top or first opening of the SPE straw.

The porous polymer matrix contained within the straw may occupy the entire length of the straw, or only a portion of the length of the straw. In one embodiment, the porous polymer monolith matrix occupies ≧half of the length of the straw, (e.g., ≧⅔, ¾, ⅘, ⅚, etc. of the straw). In one embodiment, the porous polymer matrix occupies ≦half of the length of the straw (e.g., ≦⅓, ¼, ⅕, ⅙, etc.). One end of the porous polymer matrix is generally relatively even or flush with the eluting end of the straw. However, it may be useful to have the porous polymer monolith matrix located within the straw, further from the eluting end. Generally, the porous polymer monolith matrix is not flush with the first opening or top, to allow deposition of sample within the straw. However, in some embodiment, it may be beneficial to have the porous polymer monolith matrix relatively even with the first opening or top of the straw.

### Experimental Research Enabling Multichannel Air-Pressure Driven Fluid Dispenser (ERMAF) Apparatus

In one embodiment, the components of the various straws described herein are assembled in an apparatus (automated or semi-automated) for rapid, high yield, quality purification of a plurality of samples at one time. One such apparatus is the ERMAF, described herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention can be defined in any of the following numbered paragraphs:
1. A method for purification of RNA from a sample, comprising:
   a) preparing the sample in a solution of lysis buffer and depositing the sample solution into a first end of a lysis straw such that the sample solution flows through the matrix of the lysis straw and is eluted from the opposite end of the lysis straw; and
   b) depositing the eluted material into a first end of a solid phase extraction (SPE) straw, such that the deposited solution flows through the matrix of the SPE straw towards the opposite end of the SPE straw; and
   c) eluting the RNA from the SPE straw by depositing a solution of elution buffer, into the first end of the SPE straw, such that the deposited solution flows through the matrix of the SPE straw and is eluted from the opposite end of the SPE straw, wherein purified RNA from the sample is present in the eluate of the SPE straw.
2. The method of paragraph 1, wherein, the RNA is total RNA, the sample is a cell sample; and step b) requires adding a precipitating solution to the eluted material from step a), and then depositing the solution into the first end of the solid phase extraction (SPE) straw, wherein the straw comprises silica microspheres, such that the deposited solution flows through the matrix of the SPE straw towards the opposite end of the SPE straw.
3. The method of paragraph 1, wherein the RNA is mRNA, and step b) further comprises depositing the solution into the first end of the solid phase extraction (SPE) straw, wherein the straw comprises oligo-dT.
4. The method of paragraph 1, 2 or 3, wherein at least 10 psi pressure is applied to the lysis straw and/or to the SPE straw at the first end of the straw, following solution deposition, to facilitate movement of the solution through the straw.
5. The method of paragraph 1-4, wherein from about 10-60 psi pressure is applied to the lysis straw and/or to the SPE straw at the first end of the straw, following solution deposition, to facilitate movement of the solution through the straw.
6. The method of paragraph 1-5, wherein from about 10-20 psi pressure is applied to the lysis straw and/or to the SPE straw at the first end of the straw, following solution deposition, to facilitate movement of the solution through the straw.
7. The method of paragraph 1-6, wherein the sample solution and the lysis straw is heated to about 60° C. and/or the eluted material from step a) is heated to about 60° C. for about 10 minutes, and/or the eluted material from step a) is incubated with DNAse in 1×DNAse buffer for 5-10 minutes at about 25° C., and/or the lysis straw eluate solution in step b) is heated to about 60° C. prior to deposition in the SPE straw.
8. The method of paragraph 1-7 wherein the lysis buffer is 0.1% Triton X-100, 10 mM Tris-Cl, (pH 7.5), 1 mM EDTA, 1×RNAse inhibitor.
9. The method of paragraph 1-8, wherein the loading buffer is 500 mM NaCl, 10 mM Tris-Cl (pH 7.5), 1 mM EDTA, 1×RNAse inhibitor.
10. The method of paragraph 1-9, wherein the elution buffer is 10 mM Tris-Cl, (pH 7.5), 1 mM EDTA, 1×RNAse inhibitor.
11. The method of paragraph 2, 4-10, wherein the precipitating solution of step b) is an alcohol/salt solution.
12. The method of paragraph 11, wherein the alcohol salt solution of step b) is isopropanol and ammonium acetate pH 5.2, and wherein a volume of isopropanol that is equal to the volume of the eluted material of step a) is added, and a volume of 3M ammonium acetate pH 5.2 that is ⅕^{th} the volume of the eluted material of step a) is added.
13. The method of paragraph 2, 4-12, wherein the precipitating solution of step b) is selected from the group consisting of isopropanol, ethanol, sulfolene, butanol, acetone, acetonitrile, and high salt.
14. The method of paragraph 2, 4-13, wherein following step b), and prior to step c) 70% Ethanol is added to the SPE straw and air is then passed through the straw at 60 psi.
15. An apparatus for purification of total RNA comprising, a lysis porous polymer monolith matrix contained within a first open tube, and a solid phase extraction (SPE) porous polymer monolith matrix comprising silica microspheres contained within a second open tube, the open tubes and the matrixes being able to withstand air pressure of up to 150 psi and temperatures of up to 70° C.
16. The apparatus of paragraph 15, wherein the lysis porous polymer monolith matrix has a pore size of about 3-10 micrometers, and has —COOH functionalized carbon nanotubes embedded within, and/or the SPE porous polymer monolith matrix has a pore size of about 1-5 microns, and has clusters of silica microbeads of 0.70 um embedded within.
17. An apparatus for purification of mRNA comprising, a lysis porous polymer monolith matrix contained within a first open tube, and solid phase extraction (SPE) porous polymer monolith matrix comprising oligo dT, contained within a second open tube, the open tubes and the matrixes being able to withstand up to 150 psi air pressure and temperature up to 70° C.
18. The apparatus of paragraph 17, wherein the lysis porous polymer monolith matrix has a pore size of about 3-10 micrometers, and has —COOH functionalized carbon nanotubes embedded within, and/or the SPE porous polymer monolith matrix has a pore size of about 1-5 microns, and contains oligo-dT cellulose.
19. The apparatus of paragraph 15-18, wherein the open tubes are made of a polyolefin and/or have an outer diameter of about 3.18 mm and an inner diameter of 2 mm or 1 mm, and/or a length of about 10 cm.
20. The apparatus of paragraph 15-19 wherein the first open tube and the second open tube are each connected at one end to a reservoir for delivery of solution or air to the tube.
21. The apparatus of paragraph 15-20, wherein the first open tube and the second open tube are part of the experimental research enabling multichannel air-pressure driven fluid dispenser (ERMAF).
22. A lysis straw comprising a lysis porous polymer monolith matrix contained within an open tube, wherein the porous polymer monolith matrix has a pore size of about 3-10 micrometers.
23. The lysis straw of paragraph 22, wherein the lysis porous polymer monolith matrix has —COOH functionalized multiwall hollow carbon nanotubes, with an outer diameter of 15±5 nm, and an length from 5-20 microns, embedded within.
24. A solid phase extraction (SPE) straw comprising a solid phase extraction porous polymer monolith matrix contained within an open tube, wherein the porous polymer monolith matrix has a pore size of about 1-5 microns, and has clusters of silica micro spheres of 0.70 μm embedded within.
25. A solid phase extraction (SPE) straw comprising an oligo-dT porous polymer monolith matrix contained within an open tube.
26. The lysis straw or SPE straw of paragraph 22-25, wherein the open tube and the porous polymer monolith matrix is capable of withstanding applied air pressure of up to 100 psi and temperatures of up to 70° C.
27. The lysis straw or SPE of paragraph 26, wherein the open tube is made of a polyolefin and has an outer diameter of about 3.18 mm and an inner diameter of 2 mm or 1 mm and has a length of about 10 cm.

The present invention is further illustrated by the following Examples. These Examples are provided to aid in the understanding of the invention and are not construed as a limitation thereof.

### EXAMPLES

### Example 1

### Total RNA Isolation from MDCK Cells

### Reagents and Materials:

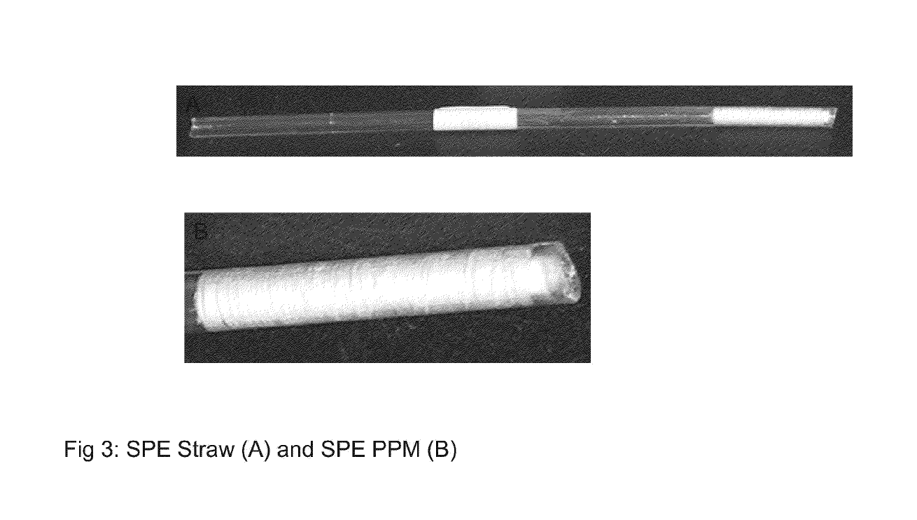

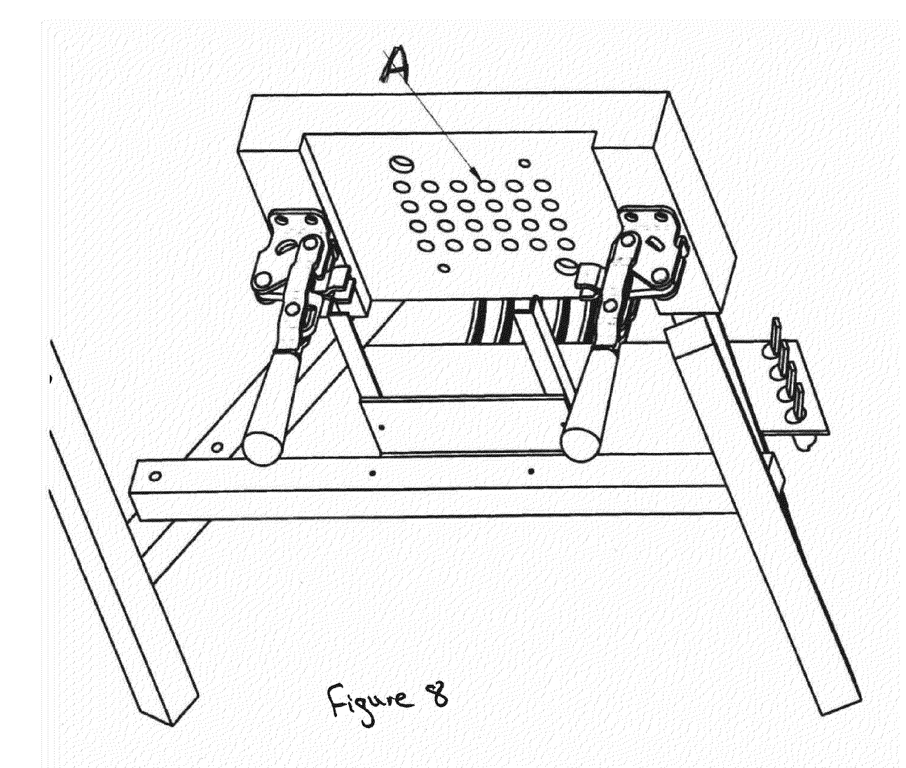

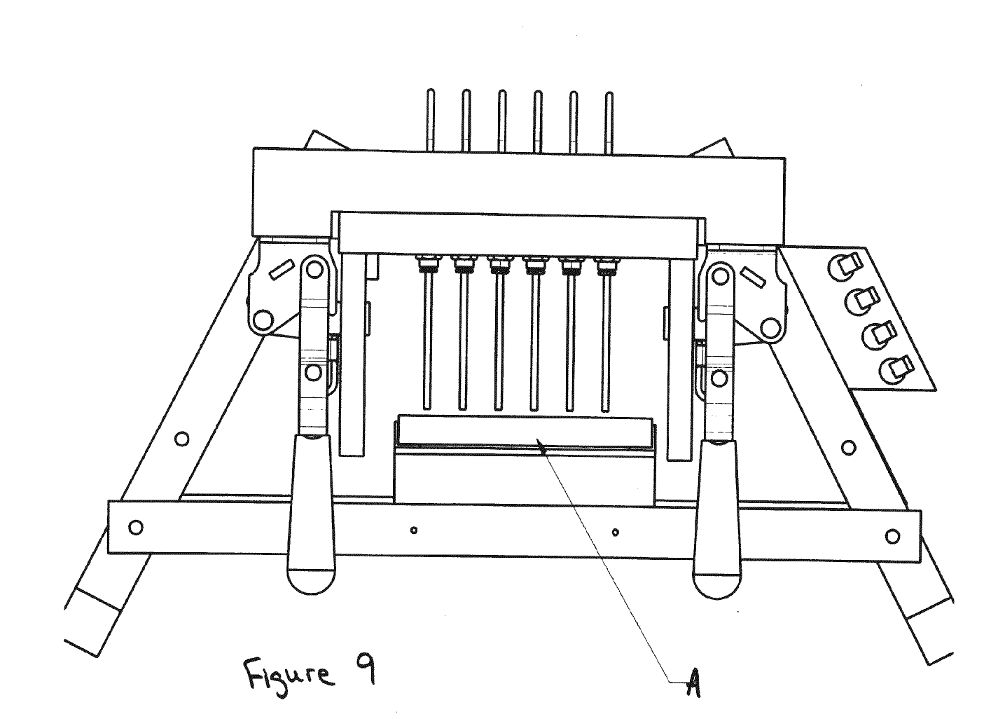

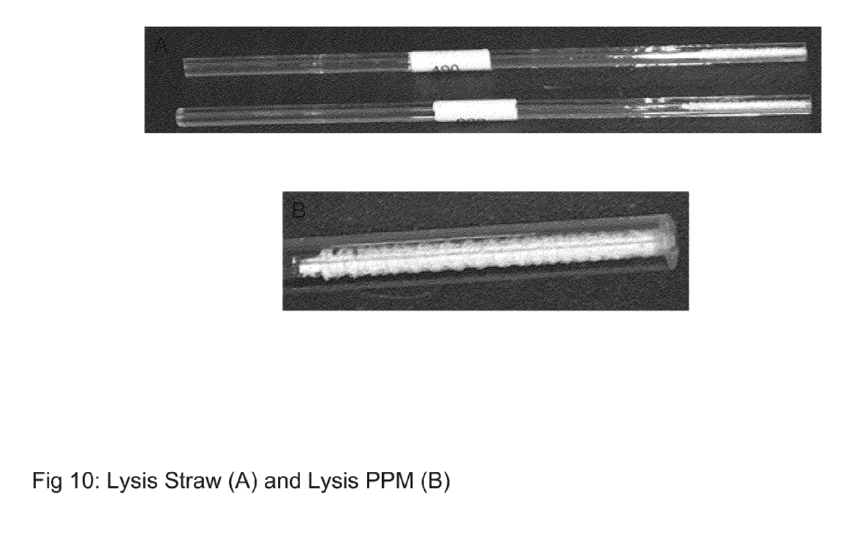

i) Mili-Q purified water, autoclaved after treatment with 0.1% DEPC (overnight stiffing) and 1×TE Solution made with DEPC treated water
ii) RNA Elution Buffer (1×TE with DEPC water+1×RNAsecure reagent from Ambion)
iii) Cell Lysis Buffer—0.1% Triton X-100+RNA Elution Buffer
iv) RNAse free DNAse (PL-DNAse from Ambion) and related buffer solution
v) 75% Ethanol made with DEPC treated water
vi) 2-Propanol
vii) 3M Ammonium Acetate Solution pH 5.2 made with DEPC treated water
viii) RNAzap solution for surface cleaning
ix) Lysis straws (FIG. 10), solid phase extraction straws (SPE100 straws) (FIG. 3), and Reservoirs
x) Straw Machine, also known as the ERMAF, discussed in more detail below (FIG. 4).
xi) Nuclease free plasticware and DEPC water treated Glassware
xii) Heater to heat up RNA Elution buffer to 60° C. and heat up plate during isolation.

### Procedure:

1. Rinse straws with 200 μl 70% Ethanol at 60 psi
2. Rinse straws with 100 μl of RNA Elution Buffer (preheated at 60 deg C.) at 60 psi
3. Load Cell samples on Lysis Straws in presence of 90 μl 0.1% Triton X-100+RNA Elution Buffer (pre heated to 60° C.)
4. Elute samples on a 96 well plate through lysis column at a pressure of 60 psi
5. Heat eluted samples on plate at 60° C. for 10 minutes
6. Cool plate down to room temperature
7. Add 5 μl DNAse and 10 μl of 10×DNAse buffer to each well
8. Incubate at room temperature for 5-10 minutes
9. Prepare SPE100 Straws between step 5 and 8. Rinse straws with 200 μl 70% Ethanol at 60 psi
10. Rinse SPE100 straws with 100 μl of RNA Elution Buffer (preheated at 60° C.) at 60 psi
11. Add 100 μl of 2-Propanol and 20 μl of 3M Ammonium Acetate pH 5.2 to each plate sample
12. Load 200 μl sample from plate on SPE100 Straws.
13. Apply 60 psi of pressure for sample to pass through SPE100 PPM
14. Wash with 200 μl of 70% Ethanol
15. Blow Dry for 1 min at 60 psi
16. Elute with 50 μl RNA elution buffer (preheated at 60° C.) at 30 psi (strop pressure and wait for 30 secs after PPM is filled with elution buffer) on a 96 well assay plate
17. Heat samples to 60° C. for 10 minutes
18. Cover plate and store at −20° C. or use for further analysis.

### Results

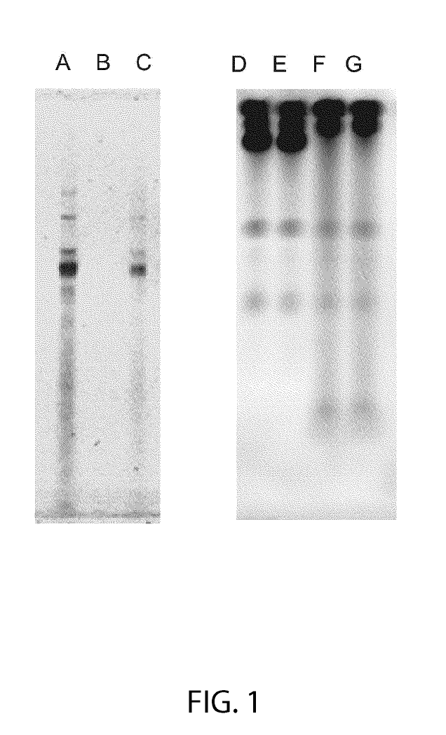

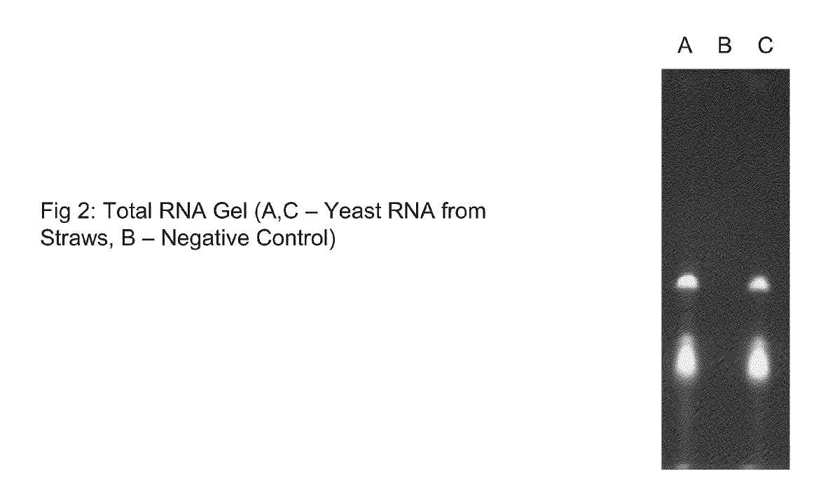

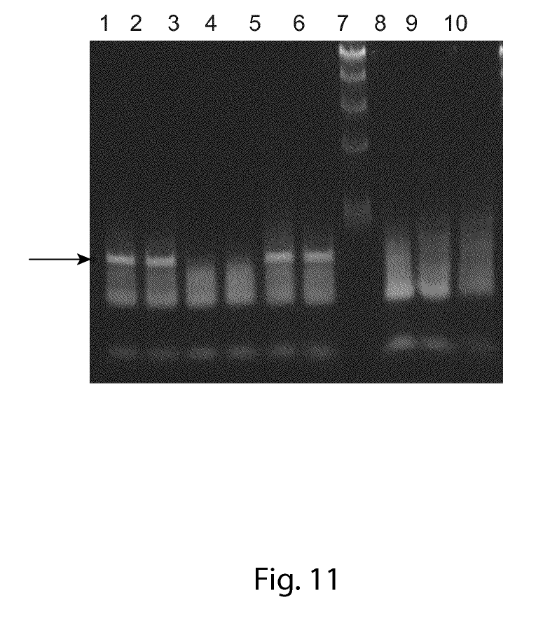

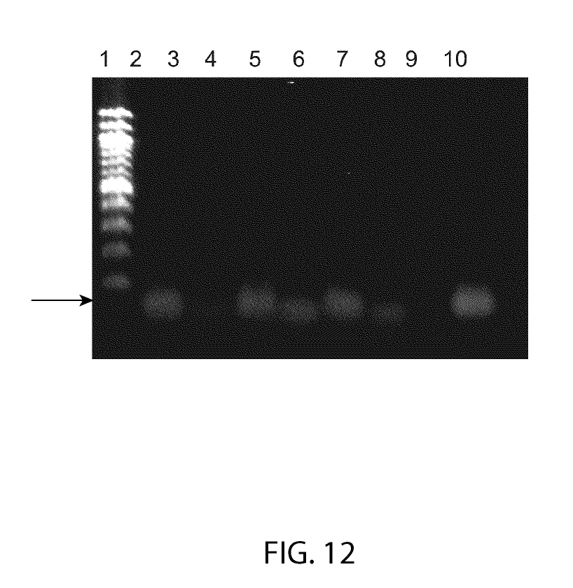

A fractionated sample of total RNA obtained from MDCK cells using this method is shown in FIG. 1. A fractionated sample of total RNA obtained from yeast using this method is shown in FIG. 2. The RNA isolated has almost no DNA contamination even without DNAse treatment. The RNA obtained is consistently intact and stable enough to use in further experiments, such as generation of cDNA. Such cDNA, generated from the total RNA obtained using the straw, porous polymer matrix isolation method is used in a PCR reaction to identify a desired sequence. The results of a PCR reaction using the cDNA are shown in FIG. 11 and FIG. 12. Yields of RNA by this technique are typically 80-90% of the RNA in the original sample.

### Discussion

This technique utilizes a novel lysis technique. The combination of a 0.1% Triton X-100 solution, the “specific “lysis straws” and the high pressure to ensure “gentle lysis” of mammalian cell membranes, but not of the nuclear membrane, thus preventing any major DNA contamination from the nucleus, at the same time extracting the majority of the RNA from the cytoplasm. This technique also utilizes a novel method of solid phase binding and enrichment of the total RNA. A combination of a specific solid phase extraction buffer and the “binding straws” containing the solid phase porous polymer monolith (PPM) can bind nucleic acids with a much higher rate. The unique manufacturing protocol of the PPM (discussed herein) ensures more surface area for nucleic acid binding throughout the length of the column (instead of the single upper surface of the regular “columns” available in the kits). This manufacturing technique together with the binding buffer and protocol, creates a much higher efficiency of RNA binding and enrichment.

The use of Air Pressure is also novel and provides significant advantages. The process uses no hazardous chemicals and is driven by air pressure, so no separate equipment like pumps, cetrifuges etc are needed. The use of relatively high air pressure together with a careful selection of buffers eliminate the presence of contaminating protein or other cellular material.

The RNA isolated has almost no DNA contamination even without DNAse treatment due to the “gentle lysis of the cells”. Protection of RNA and DNA removal: Any small amount of DNA contamination in the RNA is removed by the treatment with a RNAse free DNAse (Invitrogen), while at the same time the RNA is protected by the presence of the RNAsecure™ reagent from Ambion, all of which can be done inline in a automated instrument. The procedure is specifically designed to be easily automated, has very high yields and is fast and cheap. The process requires no hazardous chemicals and is simple enough to be handled by any technician trained in regular laboratory practices.

### Example 2

### Generation of Lysis and SPE Porous Polymer Monolyths

### Materials Used:

Butyl methacrylate (99%, BuMA), ethylene dimethacrylate (98%, EDMA), methyl methacrylate (99%, MMA), 1-dodecanol (98%), cyclohexanol (99%), benzophenone (99%, BP), and 2,2-dimethoxy-2-6 phenylacetophenone (99%, DMPAP). All were purchased from Sigma-Aldrich (St. Louis, Mo.).

Oligo-DT cellulose, RNAsecure RNase inhibitor and RNAqueous kit. All were purchased from Ambion Inc.

—COOH functionalized carbon nanotubes (CNT) were obtained from ManoLab (Newton, Mass.). Specifically, multiwall, hollow structure COOH functionalized nanotubes, having a hollow structure, with an outer diameter of 15±5 nm, length 5-20 microns, were used. These were made by the manufacturer from purified multiwall carbon nanotubes (purity >95% by TGA) which have had a reflux performed in sulfuric/nitric acid to functionalize the surfaces of the nanotubes. This process resulted in a large concentration of carboxyl (—COOH) groups on the nanotube surface, and potentially also generated other groups (e.g. —OH). After functionalization, the carboxylated nanotubes have 2-7 wt %-COOH by titration.

Silica microspheres, 0.70 μm, were obtained from Polysciences. Monodisperse silica microspheres packaged as 10% solids (w/v) aqueous suspensions were used. One of the advantages of silica microspheres over polymer microspheres is their exceptional stability. Also, the surface of silica microspheres can be easily functionalized by reaction with organosilanes. These were uniform, non-porous silica microspheres, diameters of between ˜150 nm-5 μm are available and can be used in the methods. These particles typically have size CVs of 10-15%.

RNA loading dye, RNA molecular weight standards, Oligo-dT primers, RNA gels and gel-staining dye. All were purchased from Invitrogen Corporation. A cDNA synthesis kit, purchased from Qiagen Corporation. Taqman universal PCR mastermix and custom Taqman primers, each purchased from Applied Biosystems, Inc.

### Preparation of Microspheres

a. Put up to 1000 μL of silica microsphere suspension in an eppendorf tube; make multiple tubes if necessary.
b. Centrifuge at 2500 g for 10 min
c. Decant supernatant.
d. Heat at 85° C. until silica dried to a hard pill (45 min)
e. Break up pill into a powder

### Preparation of Grafting Solution

a. Work inside fume hood!
b. Pipette MMA into grafting solution tube.
c. Melt Benzophenone in microcentrifuge tube in block at 85° C.
d. (If possible) Warm MMA in heating block
e. Pipette melted Benzophenone into grafting solution tube. Vortex.

### Preparation of Monolith Batters

Lysis Monolyth Batter (for MDK cells)

| | [μl] | % |
|---|---|---|
| BuMA | 600.0 | 15 |
| EDMA | 400.0 | 10 |
| 1-Dodec | 2100 | 52.5 |
| Cyclo | 450.0 | 11.25 |
| CNT | 450.0 | 11.25 |
| DMPAP | 45.2 | 1.13 |
| Yield | 4 ml | |

SPE 100 Monolyth Batter (AB)

| | [μl] | % |
|---|---|---|
| BuMA | 960.0 | 24 |
| EDMA | 640.0 | 16 |
| 1-Dodec | 1680.0 | 42 |
| Cyclo | 720.0 | 18 |
| DMPAP | 16.0 | 0.4 |
| Silica Susp (wet) | 4000.0 | 15 |
| Yield | 4 ml | |

Grafting Solution

| | [μl] |
|---|---|
| MMA | 3880.0 |
| Benzophenone | 120.0 |
| Yield | 4 ml |

a. Thaw Cyclohexanol and 1-Dodecanol in warm tap water
b. Work inside fume hood!
c. Melt DMPAP in microcentrifuge tube in block at 85° C.
d. Pipette BuMA, EDMA, Cyclohexanol, and 1-Dodecanol into batter tube.
e. (if possible) Warm batter tube.
f. Pipette melted DMPAP into grafting solution tube. Vortex.
g. Add silica microspheres to batter tube (must be done after all other ingredients are mixed). Suspend by sonicating for 2-3 minutes, holding the tube near the top of the liquid.

Load the Appropriate Porous Polymer Matrix into the Straws, as described in Example 3 below.

### Example 3

### Generation of the Straws for RNA Purification

The straws were made up of Zeonor 690R, a polyolefin (polymer) material from Zeon Corporation (Japan). The straws were prepared (extruded) by a local company, Phi-Tech, according to our specifications. The outer diameter was about 3.18 mm and the inner diameter was either 2 mm or 1 mm. The length of the straws was about 10 cms.

### Safety

a. Work inside fume hood, especially when using compressed air

b. If ever handling outside fume hood, wear Organic-vapor mask

c. Dispose of all wastes in hazardous material jars

### Anneal

a. Use aluminum foil as a “tablet” so that straws do not sag.

b. Use only upper tray; straws may melt if left on lower tray.

c. Leave straws in ovens (on the aluminum foil)

d. Temp=165° C.

e. Time=60 min

f. Turn oven off and let the door closed for several hours

### Load

a. Clean a bent Allen Key with Ethanol, and use it to scratch the inside of the straws for about ½ inch using a drilling machine (see FIG. 17A).
b. Sand one edge off of each straw tip.
c. Clean the inside of the straws with a long wire and air pressure
d. Put straws in a plastic bag, add Ethanol (100%), close the bag and lay it in ultrasonic bath for 10 min.

(All Following Steps are Performed Under the Fume Hood!)
e. Dry the straws using filtered air.
f. Wrap appropriate labels onto the straws, 1.5″ from end.
g. Turn the bar that will hold the straws upside down against the table.
h. Push the straws through the fitting until they bottom out against the table; tug i. out to engage the fittings.
j. Fill the bar; fill empty fittings with pegs (See FIG. 17B).

### Graft

a. Pipette grafting solution (16 μl) into straw tips [toxic—be careful] (See FIG. 17C)
b. Place bar into UV oven, with straw tips angled down (black tape strip down)
c. UV cure for 10 min (Time-1-0-0-enter-start)
d. Pour extra grafting solution into a collection dish
e. Blow the straws dry with compressed air (use the station setup), for 60 sec, 30 psi (See FIG. 17D)

### Fill

a. Agitate desired batter (e.g., SPE batter or Lysis batter) to disperse particles
b. Fill tips (160)
c. Pipette volume of batter into straw. Pipette quickly to avoid bubbles (See FIG. 17E)
d. Place bar into UV oven, with tips angled down (black tape strip down)
e. UV cure for 15 min
f. Flip bar
g. UV cure for 15 min

### Rinse

a. Cut reservoir, 1.25″ long, and push over tips of the straw/bar assembly (FIG. 17F).
b. Load bar(s) into the frame.
c. Pipette 200 μl methanol into each reservoir.
d. Load the frame into the dispenser (FIG. 17G).
e. Start Rinse at 30 psi until all methanol has dispensed.
f. Pipette 100 μl ethanol (70%) into each reservoir.
g. Start Rinse at 30 psi until all ethanol has dispensed.
h. Remove from dispenser.
i. The Straws are ready to use.

### Discussion

The straws used for lysing the cells are designed with a larger pore size (about 3-10 micrometers, about 6 micrometers on average) and have carbon nanotubes embedded in the porous polymer monolith (PPM). The carbon nanotubes are responsible for rupturing the cell membrane without destroying the nuclear membrane. The lysis buffer containing the mild detergent helps in this process. The larger pore sizes of the lysis straw PPM ensures that the cell debris and any unlysed cells do not clog up the straw and reduce the flow through the PPM. The SPE straws have smaller pore sizes (about 1-5 micrometer, about 2 micrometers on average) and have clusters of silica microbeads embedded in them.

### Example 4

### Generation of Antibody-Agarose Straws for Protein Purification

Straws can be generated with a variety of other materials for use in purification of cellular components. For example, porous polymer matrix (PPM) only can be used, or PPM+oligodT cellulose, PPM+cintered plugs, PPM+magnetic beads, Cintered plugs+oligodT cellulose, cintered plugs+silica microspheres, cintered plugs+magnetic beads, PPM+Ab-Agarose, or cintered plugs+Ab-agarose. The following is an example how to use one such type of straw that has antibody-agarose in the matrix, for protein purification.
1. Rinse straws with 200 μl PBS (Phosphate Buffer Saline, 100 mM Potassium phosphate+150 mM Sodium Chloride) at 60 psi
2. Load Cell samples on lysis Straws in presence of 90 μl 0.1% Triton X-100+Phosphate Buffer Saline+protease inhibitor cocktail
3. Elute samples on a 96 well plate through lysis column at a pressure of 60 psi
4. Prepare SPE100 Straws with PPM+Ab-Agarose, rinse straws with PBS (Phosphate Buffer Saline) at 60 psi
5. Load 200 μl sample from plate on SPE100 Straws and apply 10 psi of pressure for sample to pass through SPE100 PPM
6. Wash with 200 μl of PBS (Phosphate Buffer Saline) at 60 psi
7. Elute with 50 μl Protein elution buffer (0.2 M Glycine, pH 3±1.85) at 30 psi (stop pressure and wait for 15 secs after PPM is filled with elution buffer) collecting on a 96 well assay plate, already containing 50 μl of 2×PBS (200 mM Potassium phosphate+300 mM Sodium Chloride)
8. Add Protease inhibitor cocktail and store at 4° C.

### Example 5

### The Experimental Research Enabling Multichannel Air-Pressure-Driven Fluid Dispenser (ERMAF)

Innovative research to develop new biological sample preparation techniques are limited by simple mechanical tools. We have designed and developed an air-pressure driven mechanical device called ERMAF dispenser and complementary “sample-straws” to aid in biological sample preparation and related research. The device is suited for lysing biological cells and preparing samples like binding, washing and eluting nucleic acid samples from a solid phase support. It has been tested using a porous polymer monolith embedded sample preparation tool (straw system) on mammalian, bacterial and yeast cells to isolate nucleic acids (DNA and RNA) from cells.

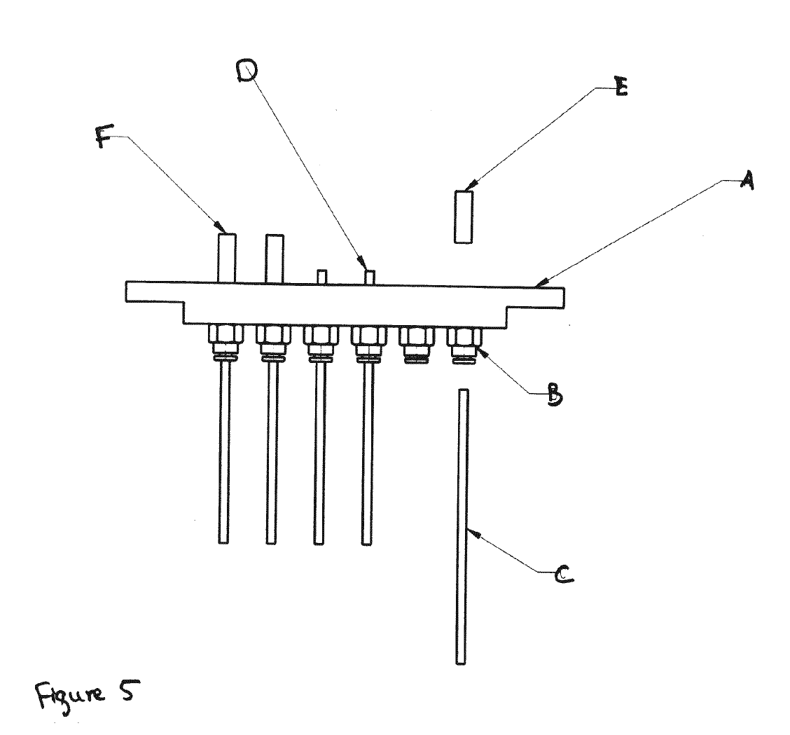

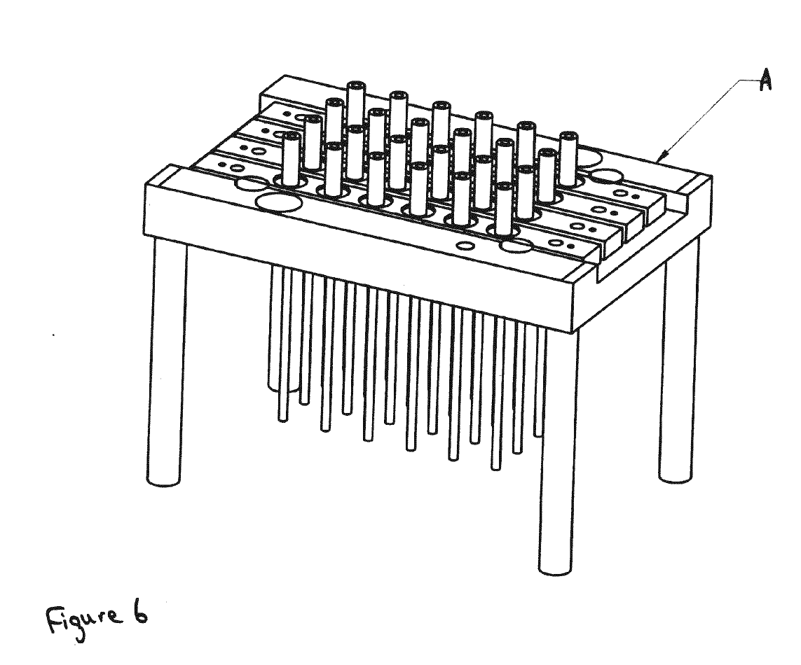

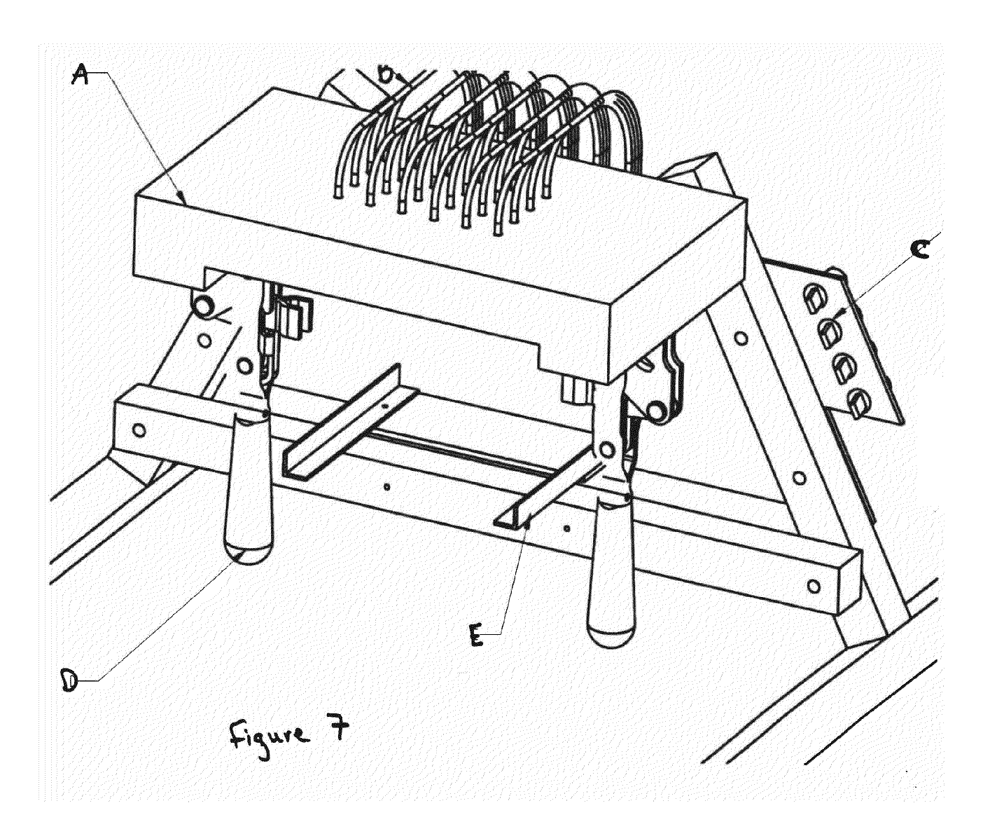

The ERMAF dispenser is a device to purify biological samples from different biological cells/tissue. The dispenser uses air pressure to drive fluids through a “straw”-like plastic tubing, containing porous polymer monolith (PPM) or other polymers (like surface-activated cellulose). The use of air pressure to drive fluids for purification of biomolecules is already used in different “lab-on-a-chip-devices” and in sample preparation techniques using “vacuum manifolds”. The unique and important differences between the vacuum manifolds and the ERMAF dispenser is simplicity of design, use and scalability as also the amount of pressure used (3-10 times more) of the ERMAF. The “straws” containing the PPM, are uniquely designed and manufactured to withstand pressures of up to 150 psi and can be used for either lysis of cells or for binding of nucleic acids (like DNA/RNA). The ERMAF dispenser and the “sample-straws” have the following innovations in itself for simple, quick and efficient handling of biological samples:
1. The straw (FIG. 5C) consists of a tube, extruded from a hard clear polymer such as Zeonex, with a length of approximately 4.0″, an outer diameter of approximately 0.13″ and an inner diameter of 0.040″ to 0.080″. The straw is filled with PPM for part or all of its length. The inner surface of the straw may be scratched with a metal tool to make the surface rough, so that the PPM will adhere better. A grafting process may also be used to form a thin primer layer inside the straw, which also promotes better PPM adhesion. The PPM may be of the large-pore type, which is used to lyse cells, or may be of the small-pore type, which contains silica and is used to bind biomolecules.
2. The straw can conveniently by inserted into a bar (FIG. 5A) containing six standard one-touch pneumatic fittings (FIG. 5B), whose rear stops have been drilled out to allow the straw to pass through the fitting and poke out the top (FIG. 5D). A reservoir is formed from a section of Teflon tubing (FIG. 5E), approximately 1.0″ long, 0.13″ inner diameter, and 0.25″ outer diameter. This piece of Teflon tubing fits snugly on top of the straw to form a cup-like reservoir (FIG. 5F) on top of the straw. The bar allows the user to handle an entire row of six straws together at once, which is convenient during the process of loading the PPM into the straws.
3. Four bars are assembled together into a frame (FIG. 6A), which holds the bars in place. The frame has legs, which allow it to stand on a table or benchtop. The reservoirs are held upright, so that the user can pipette samples or buffers into the reservoirs. The spacing of the straws matches the spacing of a standard microtiter plate; the current version has 18 mm spacing in each direction to fit a 24-well plate, but the concept could be scaled up to a 96-well or 384-well plate.
4. The dispenser consists of an aluminum pressure block (FIG. 7A), fed with pressurized air by hoses (FIG. 7B) and mounted on legs. The lower side of the aluminum manifold has an array of holes (FIG. 8A), each of which is fed by air pressure from one hose. The edges of the holes are fitted with o-rings. The flow of air through the hoses is turned on or off by a set of 4 pneumatic switches (FIG. 5C). Each switch controls the air flow to one of the 4 rows of the pressure block. The user may turn off the air pressure to rows that he/she does not wish to use.
5. To use the system, the user pipettes biological samples or buffers into the reservoirs. The user then attaches the frame to the underside of the pressure block so that the reservoirs extend up into the holes on the underside of the pressure block (FIG. 9). The o-rings on the edges of the holes form a face-seal against the surface of the bar, so that the reservoirs can be pressurized. The frame is held in place by toggle clamps (FIG. 7D) which are strong enough to resist the force of the air pressure which is applied to the straws. A microtiter plate (FIG. 9A) is placed in the mounting rails (FIG. 7E), which position the microtiter plate below the lower tips of the straws.
6. When the user flips the switches, air pressure is delivered to the pressure block and applied to the liquid in the reservoirs. This pressure forces the liquid down into the straws, through the PPM, and out into the microtiter plate. Typically, the user wishes to pass a sequence of several liquids through the straws. To do this, the user removes the frame, re-fills the reservoirs with the next liquid in the sequence, and replaces the frame.

In addition, the ERMAF device uses one-touch pneumatic fittings with the stop drilled out for an easy, quick-release way of making a seal. It uses a piece of tubing to form a simple, disposable reservoir. Scratches are made on the inside of the straw for better PPM adhesion. The frame breaks out into individual bars so that you can cure all straws. Magnets, alignment pins, toggle clamps, and frame legs are all designed for convenient handling.

Other advantages are that the ERMAF dispenser enables the researcher to develop and use a fast and reliable method for purification of biomolecules. The device is easy and cheap to manufacture. It is easy to operate and requires very little maintenance. The straws can be filled using different polymer or functionalized material for sample preparation making the ERMAF dispenser format versatile and usable for all kinds of biomolecules including nucleic acids, proteins and even carbohydrates or lipids. The simple design enables fast processing of samples and ease of switching from one type of application to another.

The straws and the ERMAF dispenser have a variety of potential applications, depending upon their composition, such as isolation of mRNA from eukaryotic cells or purification of proteins using antibody-agarose columns. ERMAF dispenser designed for high throughput analysis allows for the rapid, inexpensive screening for candidate drug molecules for clinical trials, discovery of potential drug targets in the cells by looking at up or down regulation of genes during treatment or cell stress, molecular diagnostics through DNA based microarray, and high throughput pharmacogenomics.

### Example 6

### Messenger RNA Isolation from MDCK Cells and Yeast Cells

### Reagents and Materials:

### 2× Loading Buffer:

DEPC Water

1 M NaCl (Make 2M Stock in DEPC Water and Autoclave before mixing)

20 mM Tris-Cl, pH 7.5

2 mM EDTA

2×RNAsecure reagent

### Washing Buffer (1× Loading Buffer):

DEPC Water

500 mM NaCl

10 mM Tris-Cl, pH 7.5

1 mM EDTA

1×RNAsecure reagent

### Elution Buffer:

DEPC Water

10 mM Tris-Cl, pH 7.5

1 mM EDTA

1×RNAsecure reagent

Oligo dT straws:

### Preparation of Oligo-dT Porous Polymer Monolith Matrix in Oligo-dT Straws

1. Prepare a straw using the SPE 100 monolith batter above, but without any Silica Suspension (referred to as an SPE 0 straw/batter).
2. Prepare a slurry of oligo-dT cellulose from oligo-dT cellulose powder (e.g., Ambion), suspendedin loading buffer (Tris-Cl (10 mM), EDTA (1 mM), RNAse inhibitor (e.g. RNAsecure from Ambion)).
3. Load the oligo-dT cellulose slurry onto straws and blow through at a pressure of 5 psi.
4. Repeat the process of the above step #3 until about 5 c.c. of oligo-dT cellulose has been loaded.
5. Wash the straw with loading buffer (100 μl).

### Procedure:

1. Take Cell Extract or purified total RNA samples as starting material and heat up to 60° C. for 10 minutes, heat up 2× loading buffer at 60° C. for 10 minutes also.
2. Add equal volume of 2× Loading buffer to each sample.
3. Incubate at RT for 2-3 minutes.
4. Add mixed sample to Oligo-dT straws and apply 10 psi of pressure.
5. Wash Straws with 1× Loading buffer (>=3 times the volume of sample) at 20 psi pressure.
6. Elute the Poly-A RNA with two aliquots of Elution buffer (each half the volume of initial starting material) at 10 psi pressure to isolate pure mRNA.
7. The quality and quantity of the mRNA isolated can be checked by cDNA synthesis and PCR on prepared cDNA.

### Cell Extracts can be Generated by the Following Method:

1. Rinse lysis straws (described above) with 200 μl 70% Ethanol at 60 psi
2. Rinse lysis straws with 100 μl of RNA Elution Buffer (preheated at 60 deg C.) at 60 psi
3. Load Cell samples on Lysis Straws in presence of 90 μl 0.1% Triton X-100+RNA Elution Buffer (pre heated to 60° C.)
4. Elute samples on a 96 well plate through lysis column at a pressure of 60 psi
5. Heat eluted samples on plate at 60° C. for 10 minutes
6. Cool plate down to room temperature
7. Add 5 μl DNAse and 10 μl of 10×DNAse buffer to each well
8. Incubate at room temperature for 5-10 minutes
   Reagents and Materials for generation of cell extracts:
i) Mili-Q purified water, autoclaved after treatment with 0.1% DEPC (overnight stiffing) and 1×TE Solution made with DEPC treated water
ii) RNA Elution Buffer (1×TE with DEPC water+1×RNAsecure reagent from Ambion)
iii) Cell Lysis Buffer—0.1% Triton X-100+RNA Elution Buffer iv) RNAse free DNAse (PL-DNAse from Ambion) and related buffer solution
v) 75% Ethanol made with DEPC treated water
vi) 2-Propanol
vii) 3M Ammonium Acetate Solution pH 5.2 made with DEPC treated water
viii) RNAzap solution for surface cleaning
ix) Lysis straws (FIG. 10), solid phase extraction straws (SPE100 straws) (FIG. 3), and Reservoirs
x) Straw Machine, also known as the ERMAF, discussed in more detail below (FIG. 4).
xi) Nuclease free plasticware and DEPC water treated Glassware
xii) Heater to heat up RNA Elution buffer to 60° C. and heat up plate during isolation

### Results

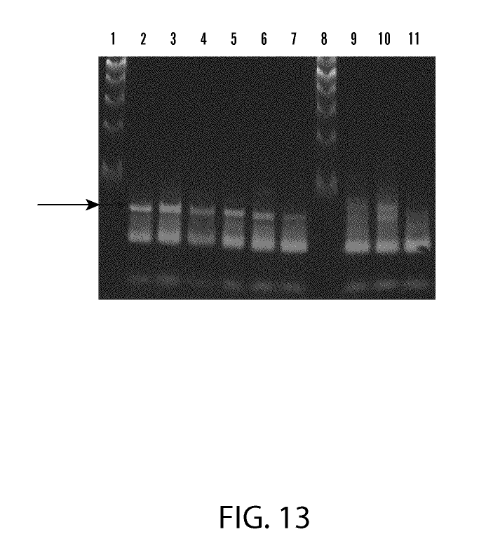

The RNA isolated has almost no DNA contamination even without DNAse treatment due to the gentle lysis of the cells (FIG. 1A-C). The messenger RNA obtained is consistently intact and stable enough to use in further experiments, such as generation of cDNA. Such cDNA, generated from the total RNA obtained using the straw, porous polymer matrix isolation method is used in a PCR reaction to identify a desired sequence. The results of a PCR reaction using the cDNA are shown in FIG. 13. Yields of RNA by this technique are typically 80-90% of the mRNA originally present in the cell sample.

### Discussion

This technique utilizes a novel lysis technique. The combination of a 0.1% Triton X-100 solution, the “specific “lysis straws” and the high pressure to ensure “gentle lysis” of mammalian cell membranes, but not of the nuclear membrane, thus preventing any major DNA contamination from the nucleus, at the same time extracting the majority of the processed mRNA from the cytoplasm.

This technique also utilizes a novel method of solid phase binding and enrichment of the mRNA. Due to a combination of a specific high salt extraction buffer and the “Oligo-dT binding straws” (FIGS. 3 A,B) containing the solid phase porous polymer monolith, the PPM can bind nucleic acids with a much higher rate. The unique manufacturing protocol of the PPM (discussed in detail below) ensures more surface area for nucleic acid binding throughout the length of the column (instead of the single upper surface of the regular “columns” available in commercially available kits). This manufacturing technique together with the unique binding buffer and protocol, creates a much higher efficiency of mRNA binding and enrichment.

The use of Air Pressure is also novel and provides significant advantages. The process uses no hazardous chemicals and is driven by air pressure through a “straw driver instrument (FIGS. 4 A, B, and C), so no separate equipment like pumps, centrifuges etc are needed. The use of relatively high air pressure together with a careful selection of buffers eliminate the presence of contaminating protein or other cellular material.

The RNA isolated has almost no DNA contamination even without DNAse treatment due to the “gentle lysis of the cells” (FIG. 1A-C). Protection of RNA and DNA removal: Any small amount of DNA contamination in the RNA is removed by the treatment with a RNAse free DNAse (Invitrogen), while at the same time the RNA is protected by the presence of the RNAsecure™ reagent from Ambion, all of which can be done inline in an automated instrument. The procedure is specifically designed to be easily automated, has very high yields. The highest expected yield for mRNA isolated is about 99% of the mRNA originally present in the cell sample, with typical yields of about 80-90%. The method is fast and cheap, process requires no hazardous chemicals and is simple enough to be handled by any technician trained in regular laboratory practices.

### Example 7

### Re-Purification of Flu RNA Using the PPM Filled Straws

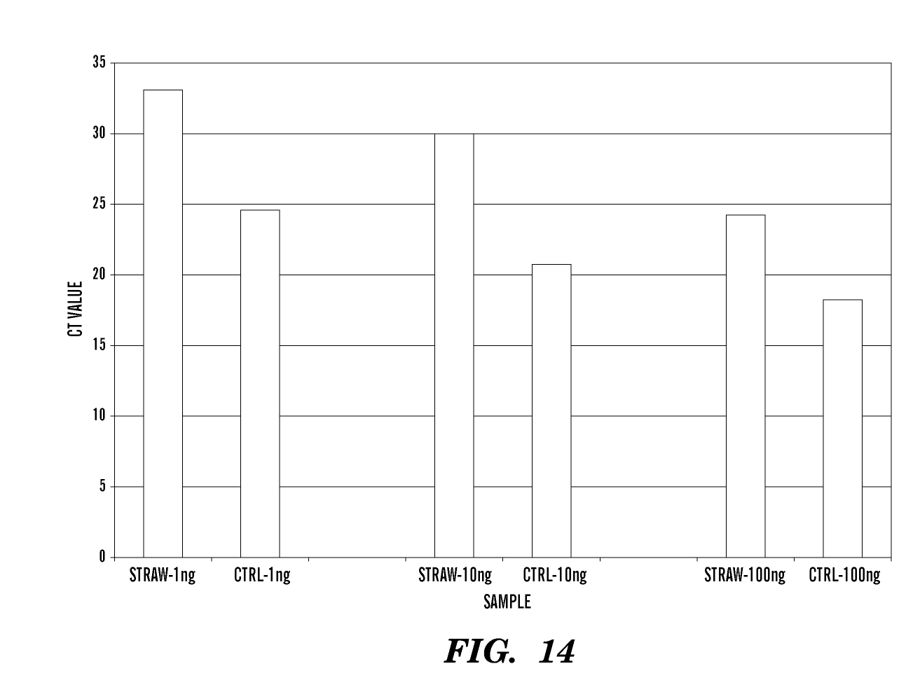

The porous polymer monolith filled straws have been used for Influenza B RNA isolation. The samples used were RNA purified from mammalian cell lysates infected with human influenza H3N1 virus. In the initial process, the RNA had been isolated from the cell lysates by Qiagen RNA isolation kits. However, the RNA contained genomic DNA contaminations from the host cell, and the RNA has been re-purified using the PPM based straw system and methods described herein. The results are shown in FIG. 14. In addition to re-purify and cleanup Flu RNA, efforts are underway to isolate the Flu RNA directly from mammalian cell lysates. The figure represents the Ct values of samples purified from RNA based cell lysates—“Ctrl”—represents Ct values of RT-PCR result from unpurified RNA and “Straw”—represents Ct values of RT-PCR results from repurified cleaned up samples.

A significant improvement in RNA extraction efficiency using the herein described methods over the commercially available Ambion kit was also observed. A statistically significant higher efficiency was observed when purifying 100 ng, 1 ng, and 0.1 ng RNA, as measured by Ct value for RT-PCR of obtained RNA products.

### Example 8

### Repeatability of Fluid Flow through PPM Based Straws

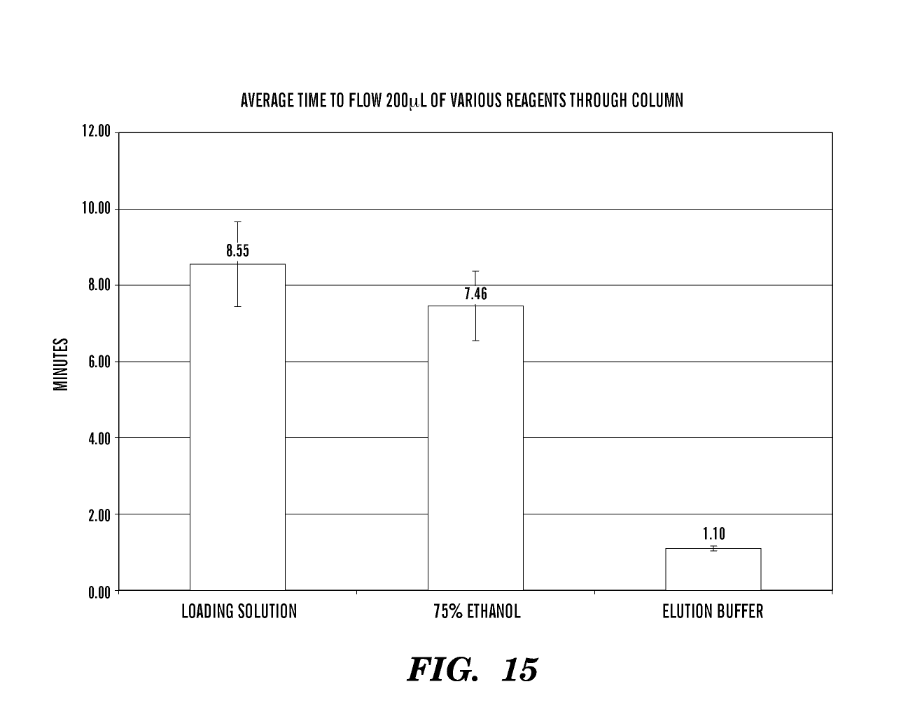

To assess the reproducibility of the PPM flow rates, the time for various fluids to traverse the straw on a set of 24 different SPE channels made in 2 different batches (FIG. 15) was measured. FIG. 15 shows the average time to flow 200 μl of the indicated reagents through the column. The variation in time between the different porous polymer monoliths was largest for the loading solution (10 ng B. subtilis genomic DNA, 3M GuSCN, 50% isopropanol) with a CV of 13%, followed by that of the 75% ethanol (CV=12%), with the elution buffer (1×TE) having a CV of 0.6%. From this set of experiments the variation in flow rate was found to be a function of the length of the PPM column and the liquid reagent.

### Example 9

### Comparison of Mammalian Cell (MDCK) RNA Isolation Using Ambion Kit Versus PPM Based Straws

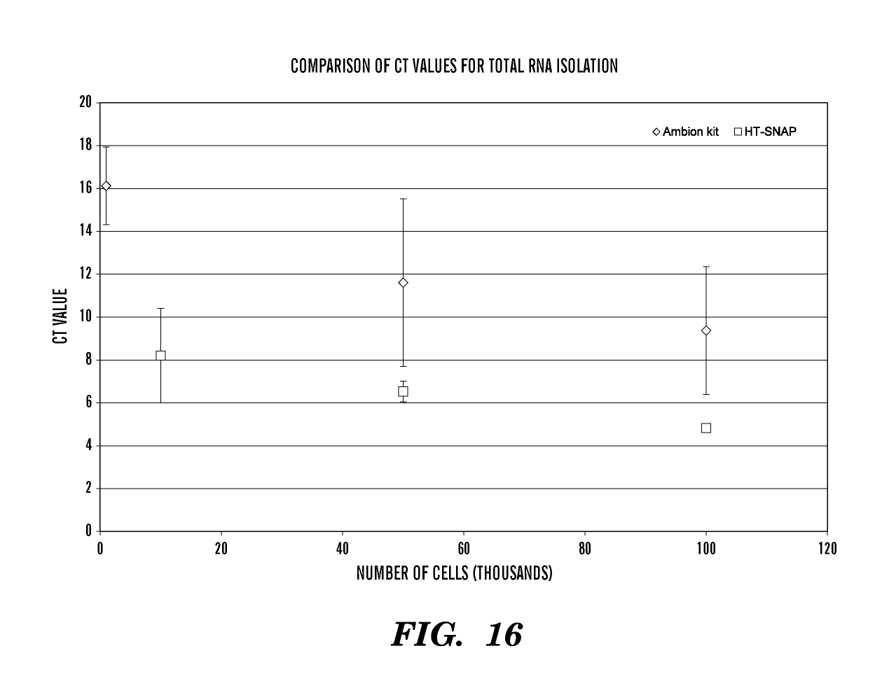

Concentration of total RNA isolated from 1000 (1), 10,000 (2), 50,000 (3) and 100,000 (4) MDCK cells using the lysis and SPE straw based method described herein, compared to the total RNA isolated from same number of cells using the Ambion kit, measured on a nanodrop UV-spectrophotometer. Each data point represents averages of 5-6 replicates; error bars represent standard deviation from the average. FIG. 16 shows the CT value of PCR products performed on cDNA synthesized from MDCK total RNA isolated using the herein described method (HT-SNAP) compared to the Ambion kit. Each data point represents averages of 3-10 replicates; error bars represent standard deviation from the average. Data presented for the 1000 cells consists of results only obtained with the Ambion kit, and for the 10,000 cells consists of results only obtained with the lysis/SPE straws.

TABLE 1

| Kit Name | Company | Purpose | Technology | Throughput | |
|---|---|---|---|---|---|
| miRNeasy 96 Kit | Qiagen | 96-well | Organic Solvent | 96 | samples |
| (Qiazol) | | purification of | Based | | |
| | | microRNA and | | | |
| | | total RNA from | | | |
| | | tissues and cells | | | |
| PAXgene 96 Blood | Qiagen | High-throughput | Silica Based Solid- | 96 | samples |
| RNA Kit | | purification of | Phase Extraction | | |
| | | cellular RNA | | | |
| | | from whole blood | | | |
| MagAttract RNA Cell | Qiagen | High-throughput | Magnetic Beads | 48 | samples |
| Mini M48 Kit | | purification of | | | |
| | | cellular RNA | | | |
| | | from cultured | | | |
| | | cells | | | |
| RNAqueous ®-96 Kit | Ambion | High-throughput | Silica Based Solid- | 96 | samples |
| | | total RNA | Phase Extraction | | |
| | | isolation from | | | |
| | | Cultured cells, | | | |
| | | Tissue etc | | | |
| MagMAX ™-96 | Ambion | High-throughput | Magnetic Beads | 96 | samples |
| Blood RNA Isolation | | purification of | | | |
| Kit | | cellular RNA | | | |
| | | from whole blood | | | |
| MicroPoly(A)Purist ™ | Ambion | mRNA isolation | Oligo-DT Cellulose | 6 | samples |
| Kit | | from cultured | Beads | | |
| | | cells, Tissue etc | | | |
| Trizol Reagent | Invitrogen | Total RNA from | Organic Solvent | 24 | samples |
| | | bacteria, Plant | Based | | |
| | | cells and Yeast | | | |
| Dynabeads mRNA | Invitrogen | mRNA from | Oligo-DT based | 20 | samples |
| Purification Kit | | Cultured cells, | Beads | | |
| | | tissue etc | | | |
| Purelink Total RNA | Invitrogen | Total RNA from | Silica Based Solid- | 10-50 | samples |
| Purification Kits | | Cultured Cells, | Phase Extraction | | |
| | | Blood, Formalin | | | |
| | | Fixed Tissue etc | | | |
| Ribominus | Invitrogen | Targeted | By selective | 6 | samples |
| | | Transcriptome | depletion of large | | |
| | | from Cultured | rRNA transcripts | | |
| | | cells or Tissue | from total RNA | | |
| SV Total RNA | Promega | Total RNA | Silica Based Solid- | 10 to 20 | samples |
| Isolation | | isolation from | Phase Extraction | | |
| | | cultured cells, | | | |
| | | tissues and Blood | | | |
| PolyATract system | Promega | mRNA isolation | Magnetic Beads | 10 to 20 | samples |
| 1000 | | from cultured | | | |
| | | cells, tissues, | | | |
| | | Blood, Yeast, | | | |
| | | Plants etc | | | |
| RNAdvance Cell or | Agencourt | Total RNA | Silica Based Solid- | 10 to 20 | samples |
| Tissue Kits | (Beckman) | isolation from | Phase Extraction | | |
| | | cultured cells, | | | |
| | | tissues and Blood | | | |

TABLE 2

| Instrument | Company | Purpose | Technology | Throughput | |
|---|---|---|---|---|---|
| iPrep | Invitrogen | Automated DNA/ | Solid-Phase | 12 | samples |
| | | Total RNA isolation | Extraction on | | |
| | | from Blood or other | automated | | |
| | | biological fluids | platform | | |
| Magna Sep | Invitrogen | Automated DNA/ | Magnetic | 8 to 96 | samples |
| | | Total RNA isolation | Separator on | | |
| | | from Tissue, Cultured | pipettes | | |
| | | Cells Blood or other | | | |
| | | biological fluids | | | |
| Maxwell | Promega | DNA, RNA (or | Solid phase | 16 | samples |
| | | Protein) purification | extraction | | |
| | | through specialized | | | |
| | | kits | | | |
| ABI6100 | Applied | DNA/RNA isolation | Solid Phase | 96 | samples |
| | Biosystems | using ABI kits | Extraction | | |
| QIACube | Qiagen | Robotic workstation | | | |
| | | based DNA, RNA, or | | | |
| | | protein purification | | | |
| | | using QIAGEN spin- | | | |
| | | column kits | | | |
| Epmotion 5075 | Eppendorf | Robotic Workstation | Solid Phase | 96 | Samples |
| | | based DNA/RNA | Extraction | | |
| | | purification using | | | |
| | | Qiagen RNAeasy Kit | | | |
| BioMek | Beckman | Robotic Workstation | Solid Phase | 96 or 384 | samples |
| | Coulter | based DNA/RNA | Extraction | | |
| | | purification using | | | |
| | | Agencourt DNA/ | | | |
| | | RNA isolation kits | | | |
| X-Tractor | Corbett Life | Automated DNA/ | Solid Phase | 96 | Samples |
| | Sciences | Total RNA isolation | Extraction | | |
| | | from Tissue, Cultured | | | |
| | | Cells Blood or other | | | |
| | | biological fluids | | | |
| Lab Turbo 36 | Taigen | Automated DNA/ | Solid Phase | 36 | Samples |
| | Bioscience | Total RNA isolation | Extraction | | |
| | | from Tissue, Cultured | | | |
| | | Cells Blood or other | | | |
| | | biological fluids | | | |
| Lab Turbo 496 | Taigen | Automated DNA/ | Solid Phase | 384 | Samples |
| | Bioscience | Total RNA isolation | Extraction | | |
| | | from Tissue, Cultured | | | |
| | | Cells Blood or other | | | |
| | | biological fluids | | | |

What is claimed:

1. A method for purification of RNA from a sample, comprising:
a) preparing the sample in a solution of lysis buffer and depositing the sample solution into a first end of a lysis straw such that the sample solution flows through the matrix of the lysis straw and is eluted from the opposite end of the lysis straw; and
b) depositing the eluted material into a first end of a solid phase extraction (SPE) straw, such that the deposited solution flows through the matrix of the SPE straw towards the opposite end of the SPE straw; and
c) eluting the RNA from the SPE straw by depositing a solution of elution buffer, into the first end of the SPE straw, such that the deposited solution flows through the matrix of the SPE straw and is eluted from the opposite end of the SPE straw, wherein purified RNA from the sample is present in the eluate of the SPE straw.

2. The method of claim 1, wherein, the RNA is total RNA, the sample is a cell sample; and step b) requires adding a precipitating solution to the eluted material from step a), and then depositing the solution into the first end of the solid phase extraction (SPE) straw, wherein the straw comprises silica microspheres, such that the deposited solution flows through the matrix of the SPE straw towards the opposite end of the SPE straw.

3. The method of claim 2, wherein at least 10 psi pressure is applied to the lysis straw and/or to the SPE straw at the first end of the straw, following solution deposition, to facilitate movement of the solution through the straw.

4. The method of claim 3, wherein the sample solution and the lysis straw is heated to about 60° C. and/or the eluted material from step a) is heated to about 60° C. for about 10 minutes, and/or the eluted material from step a) is incubated with DNAse in 1×DNAse buffer for 5-10 minutes at about 25° C., and/or the lysis straw eluate solution in step b) is heated to about 60° C. prior to deposition in the SPE straw.

5. The method of claim 4, wherein the lysis buffer is 0.1% Triton X-100, 10 mM Tris-Cl, (pH 7.5), 1 mM EDTA, 1×RNAse inhibitor.

6. The method of claim 1, wherein the RNA is mRNA, and step b) further comprises preparing the eluted material in a loading buffer which is deposited into the first end of the SPE straw, wherein the SPE straw comprises oligo-dT.

7. The method of claim 6, wherein at least 10 psi pressure is applied to the lysis straw and/or to the SPE straw at the first end of the straw, following solution deposition, to facilitate movement of the solution through the straw.

8. The method of claim 7, wherein the sample solution and the lysis straw is heated to about 60° C. and/or the eluted material from step a) is heated to about 60° C. for about 10 minutes, and/or the eluted material from step a) is incubated with DNAse in 1×DNAse buffer for 5-10 minutes at about 25° C., and/or the lysis straw eluate solution in step b) is heated to about 60° C. prior to deposition in the SPE straw.

9. The method of claim 6, wherein from about 10-20 psi pressure is applied to the lysis straw and/or to the SPE straw at the first end of the straw, following solution deposition, to facilitate movement of the solution through the straw.

10. The method of claim 6, wherein the loading buffer is 500 mM NaCl, 10 mM Tris-Cl (pH 7.5), 1 mM EDTA, 1×RNAse inhibitor.

11. The method of claim 6, wherein the elution buffer is 10 mM Tris-Cl, (pH 7.5), 1 mM EDTA, 1×RNAse inhibitor.

12. The method of claim 1, wherein the precipitating solution of step b) is isopropanol and ammonium acetate pH 5.2, and wherein a volume of isopropanol that is equal to the volume of the eluted material of step a) is added, and a volume of 3M ammonium acetate pH 5.2 that is ⅕^{th} the volume of the eluted material of step a) is added.

13. An apparatus for purification of total RNA comprising, a lysis porous polymer monolith matrix contained within a first open tube, and a solid phase extraction (SPE) porous polymer monolith matrix comprising silica microspheres contained within a second open tube, the open tubes and the matrixes being able to withstand air pressure of up to 150 psi and temperatures of up to 70° C.

14. The apparatus of claim 13, wherein the lysis porous polymer monolith matrix has a pore size of about 3-10 micrometers, and has —COOH functionalized carbon nanotubes embedded within, and/or the SPE porous polymer monolith matrix has a pore size of about 1-5 microns, and has clusters of silica microbeads of 0.70 um embedded within.

15. The apparatus of claim 14, wherein the open tubes are made of a polyolefin and/or have an outer diameter of about 3.18 mm and an inner diameter of 2 mm or 1 mm, and/or a length of about 10 cm.

16. The apparatus of claim 15, wherein the first open tube and the second open tube are each connected at one end to a reservoir for delivery of solution or air to the tube.

17. The apparatus of claim 16, wherein the first open tube and the second open tube are part of the experimental research enabling multichannel air-pressure driven fluid dispenser (ERMAF).

18. An apparatus for purification of mRNA comprising, a lysis porous polymer monolith matrix contained within a first open tube, and solid phase extraction (SPE) porous polymer monolith matrix comprising oligo dT, contained within a second open tube, the open tubes and the matrixes being able to withstand up to 150 psi air pressure and temperature up to 70° C.

19. The apparatus of claim 18, wherein the lysis porous polymer monolith matrix has a pore size of about 3-10 micrometers, and has —COOH functionalized carbon nanotubes embedded within, and/or the SPE porous polymer monolith matrix has a pore size of about 1-5 microns, and contains oligo-dT cellulose.

20. The apparatus of claim 19, wherein the open tubes are made of a polyolefin and/or have an outer diameter of about 3.18 mm and an inner diameter of 2 mm or 1 mm, and/or a length of about 10 cm.

21. The apparatus of claim 20, wherein the first open tube and the second open tube are each connected at one end to a reservoir for delivery of solution or air to the tube.

22. The apparatus of claim 21, wherein the first open tube and the second open tube are part of the experimental research enabling multichannel air-pressure driven fluid dispenser (ERMAF).

23. A solid phase extraction (SPE) straw comprising a solid phase extraction porous polymer monolith matrix contained within an open tube, wherein the porous polymer monolith matrix has a pore size of about 1-5 microns, and has clusters of silica micro spheres of 0.70 μm embedded within.
